(19) United States
(12) Patent Application Publication
(10) Pub. No.: US 2010/0249184 A1
(43) Pub. Date: Sep. 30, 2010

(54) INDUCTION AND/OR MAINTENANCE OF TUMOR DORMANCY BY DISRUPTION OF UROKINASE PLASMINOGEN ACTIVATOR RECEPTOR-INTEGRIN INTERACTION

(75) Inventors: Liliana Ossowski, Forest Hills, NY (US); Pratima Chaurasia, New York, NY (US); Julio A. Aguirre-Ghiso, Niskayuna, NY (US); Ming-Ming Zhou, Old Greenwich, CT (US); Michael Ploug, Copenhagen N (DK)

Correspondence Address: NIXON PEABODY LLP - PATENT GROUP, 1100 CLINTON SQUARE, ROCHESTER, NY, 14604, US

(73) Assignees: MOUNT SINAI SCHOOL OF MEDICINE, New York, NY (US); COPENHAGEN HOSPITAL CORPORATION, Copenhagen K (DK)

(21) Appl. No.: 12/531,502

(22) PCT Filed: Mar. 17, 2008
(86) PCT No.: PCT/US08/57232

§ 371 Date: Jun. 2, 2010

Related U.S. Application Data
(60) Provisional application No. 60/895,182, filed on Mar. 16, 2007.

(51) Int. Cl. A61K 31/47; A61K 31/4402; C12N 5/09; G01N 33/574; G06G 7/60

(52) U.S. Cl. 514/313; 514/357; 435/375; 435/7.23; 703/11

(57) ABSTRACT

The present invention relates to a method of inducing cancer cells into dormancy and treating cancer in a subject. This method involves administering to a subject an effective amount of a compound that disrupts interaction between urokinase plasminogen activator receptor and integrin, thereby including cancer cells into dormancy and treating cancer in the subject. Also disclosed is a method of disrupting interaction between integrin and urokinase plasminogen activator receptor on cancer cells. The present invention also relates to a method of screening for compounds effective in inducing tumor dormancy and treating cancer in a subject.

This application claims the priority benefit of U.S. Provisional Patent Application Ser. No. 60/895,182, filed Mar. 16, 2007, which is hereby incorporated by reference in its entirety.

### FIELD OF THE INVENTION

The present invention relates to the induction and/or maintenance of tumor dormancy by disruption of urokinase plasminogen activator receptor-integrin interaction.

### BACKGROUND OF THE INVENTION

In a large proportion of patients that undergo surgical removal of primary cancer and who, at the time of surgery, show no detectable disseminated disease, the cancer recurs, sometimes more than decades later. This means that small numbers of cancer cells, undetected by currently available techniques can persist in a dormant state. The mechanisms that allow them to remain dormant and alive, as well as those causing them to initiate proliferation and form overt metastases, are largely unknown.

A model of human head and neck carcinoma, which retained its partial dependence on extracellular matrix for proliferative signals in vivo was previously reported (Aguirre-Ghiso et al., Mol. Biol. Cell 12:863-879 (2001); Aguirre et al., J. Cell Biol. 147:89-104 (1999)). These cells express very high levels of both the urokinase plasminogen activator (“uPA”) and its receptor (“uPAR”), which cause the activation of the α5β1-integrin, and by recruiting epidermal growth factor, initiate a signaling cascade that leads to a persistently high level of extracellular signal-regulated kinase (“ERK”) and tumorigenicity (Liu et al., Cancer Cell 1:445-457 (2002)). It has been shown that reduction of uPAR levels through stable uPAR-antisense expression forces cells into a state of prolonged dormancy (Aguirre-Ghiso et al., Mol. Biol. Cell 12:863-879 (2001); Yu et al., J. Cell Biol. 137:767-777 (1997); Aguirre-Ghiso et al., Cancer Res. 63:1684-1695 (2003); Kook et al., EMBO J. 13:3983-3991 (1994)).

Although uPAR is linked through a GPI-anchor to the surface of cells (Ploug et al., J. Biol. Chem. 266:1926-1933 (1999)), it has been shown to possess signaling properties (Blasi et al., Nat. Rev. Mol. Cell. Biol. 3:932-943 (2002); Ossowski et al., Curr. Opin. Cell Biol. 12:613-620 (2000); Chapman, Curr. Opin. Cell Biol. 9:714-724 (1997)). Since uPAR has no cytoplasmic domain, it must be signaling by partnering with other “competent” receptors. Several such proteins, belonging to different families, such as internalization receptors (LRP, uPARAP) (Behrendt, Biol. Chem. 385:103-136 (2004); Webb et al., J. Biol. Chem. 274:7412-7420 (1999)) or growth factor receptors (Liu et al., Cancer Cell 1:445-457 (2002); Kiyan et al., EMBO J. 24:1787-1797 (2005)) have been identified. Among the interacting partners, integrins belonging to at least 3 families, β1, β2, and β3 (Aguirre-Ghiso et al., J. Cell Biol. 147:89-104 (1999); Wei et al., Science 273:1551-1555 (1996); Xue et al., Cancer Res. 57:1682-1689 (1997); Pluskota et al., Blood 101:1582-1590 (2003)) have been identified as potential signaling uPAR-partners. These latter interactions have been shown to alter a variety of functions, including phagocytosis, adhesion, migration, protease secretion (Blasi et al., Nat. Rev. Mol. Cell. Biol. 3:932-943 (2002); Ossowski et al., Curr. Opin. Cell Biol. 12:613-620 (2000); Chapman, Curr. Opin. Cell Biol. 9:714-724 (1997)), and proliferation.

Until recently, the uPAR and integrin interactions have been gleaned from co-immunoprecipitation experiments, FRET analysis, and co-localization by immunocytochemistry. The integrin (αVβ3) structure has been solved recently (Xiong et al., Science 294:339-345 (2001)), facilitating the mapping of the interaction sites with uPAR on both the α and β subunits of several integrins (Zhang et al., J. Cell Biol. 163:177-188 (2003); Wei et al., J. Cell Biol. 168:501-511 (2005); Simon et al., J. Biol. Chem. 275:10228-10234 (2000)). Equivalently reliable data for the site on uPAR involved in integrin interaction were missing, in part because until very recently (Llinas et al., EMBO J. 24:1655-1663 (2005); Huai et al., Science 311:656-9 (2006)), the structure of uPAR has not been solved.

It has previously been shown that treatment of cancer cells with a monoclonal antibody (R2) directed to an epitope located in domain III of uPAR, blocked activation of α5β1-integrin by uPAR and strongly reduced signaling to ERK (Aguirre-Ghiso et al., Cancer Res. 63:1684-1695 (2003)). This suggested domain III as a plausible site for interaction with integrin.

Because uPAR is over-expressed in many malignant tumors (Andreasen et al., Int. J. Cancer 72:1-22 (1997); Sidenius et al., Cancer Metastasis Rev. 22:205-222 (2003)), and because it was shown that the activating event between uPAR and integrin occurs only in cancer cells that express high uPAR levels, this interaction is considered to predominate in cancer and, therefore, constitutes a potentially unique target for cancer therapy. A successful intervention to disrupt this interaction might induce tumor dormancy.

The present invention is directed to overcoming these and other limitations in the art.

### SUMMARY OF THE INVENTION

One aspect of the present invention is directed to a method of inducing cancer cells into dormancy and treating cancer in a subject. This method involves administering to a subject an effective amount of a compound that disrupts interaction between urokinase plasminogen activator receptor and integrin, where the compound has the following formula:

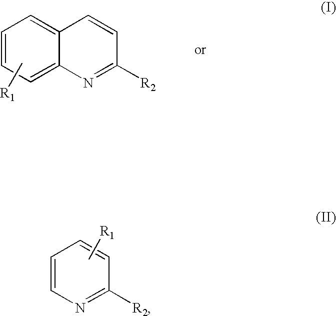

where:

R₂ is

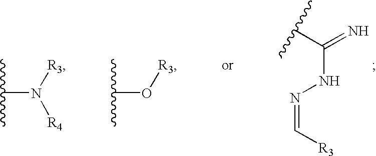

R₃ is

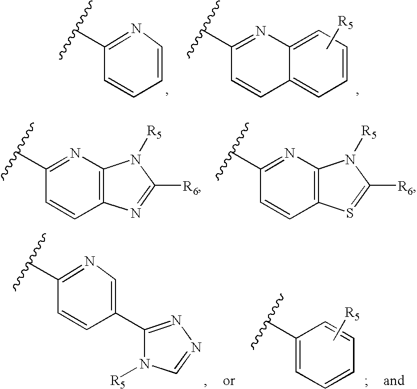

R₁, R₄, R₅, and R₆ are independently selected from the group consisting of hydrogen, —(CH₂)ₙOH, —(CH₂)ₙNH₂, —(CH₂)ₙNHCH₃, —(CH₂)ₙNHCH₂CH₃, —(CH₂)ₙ—COOH, halo, lower alkyl, haloalkyl, —(CH₂)ₙSH, lower thioalkyl, —CN, lower alkoxy, and

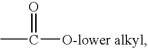

where n is an integer from 0 to 4, and pharmaceutically acceptable salts thereof, thereby inducing cancer cells into dormancy and treating cancer in the subject.

Another aspect of the present invention is directed to a method of disrupting interaction between integrin and urokinase plasminogen activator receptor on cancer cells. This method involves contacting cancer cells with an effective amount of a compound as described above.

A further aspect of the present invention is directed to a method of screening for compounds effective in inducing tumor dormancy and treating cancer in a subject. This method involves providing one or more candidate compounds. The one or more candidate compounds are contacted with urokinase plasminogen activator receptor under conditions effective to disrupt interaction between integrin and the urokinase plasminogen activator receptor. Candidate compounds which disrupt interaction between integrin and urokinase plasminogen activator receptor are identified as compounds potentially effective in inducing tumor dormancy and treating cancer in a subject.

Described herein is a series of experiments that include a test of binding interactions between purified α5β1-integrin and short synthetic uPAR-derived peptides, as well as full-length soluble recombinant uPAR (“suPAR”), with individual amino acid substitutions. These experiments led to the identification of a sequence within domain III of uPAR that is important for interaction with the integrin. Substituting one of these residues (S245) with alanine impaired the ability of uPAR to enter into functional interactions with the integrin, thereby inhibiting signaling and growth in vivo. The relevance of this binding site, and of the lateral uPAR/α5β1-integrin interaction, to ERK pathway activation and tumor growth implicates it as possible specific target for cancer therapy.

The present invention is directed to compounds that are demonstrated to have ERK inhibitory activity and have the ability to disrupt uPAR-integrin interaction. Based on this activity, these compounds are effective in inducing cancer cells into dormancy and treating cancer in a subject.

### BRIEF DESCRIPTION OF THE DRAWINGS

FIGS. 1A-H show the interaction between α5β1-integrin and immobilized uPAR and uPAR-derived synthetic peptides through solid-phase dot-ELISA. FIG. 1A shows binding of α5β1-integrin to suPAR and fibronectin. Purified suPAR, human fibronectin, or bovine serum albumin (BSA) (as a negative control) were immobilized on nitrocellulose at the indicated concentrations as described in the Examples and incubated with purified α5β1-integrin (750 ng/ml). Bound integrin was detected with anti-α5 integrin antibody followed by goat anti-rabbit IgG (H+L) HRP conjugated secondary antibody and developed with enhanced chemiluminescence (“ECL”). FIG. 1B is a graph showing that binding of integrin to suPAR is saturable. SuPAR (1.5 μg) was immobilized on nitrocellulose membrane in triplicates, incubated with 0, 0.1, 0.5, 1, 2, and 4 μg/ml of α5β1-integrin and tested for integrin binding as in FIG. 1A. The dots were scanned with NIH Image. Mean and SE are shown. FIG. 1C shows binding of α5β1-integrin to suPAR peptides. Peptides representing regions located in each of the three uPAR-domains were immobilized, incubated with α5β1-integrin, and analyzed as in FIG. 1A. Only peptide 240-248 binds α5β1-integrin. FIG. 1D is a graph showing that peptide 240-248 inhibits integrin binding to suPAR. The α5β1-integrin (750 ng/ml) was pre-incubated for 15 min at room temperature with 100 fold molar excess of 240-248 or 17-24 peptides, or with no peptide (Control) and the mixtures were applied to immobilized suPAR (1.5 μg), 2 dots per mixture, and developed as in FIG. 1A. The dots were scanned with NIH Image. The results (bars) show % of control, the lines show range. FIG. 1E is a graph showing binding of α5β1- and α3β1-integrin to suPAR and peptide 240-248. Dot-ELISA was performed in duplicate as in FIG. 1A, except that purified α5β1-integrin and α3β1-integrin (750 ng/ml) were used. Bound integrins were detected with the appropriate previously titrated antibodies followed by goat anti-rabbit IgG (H+L) HRP conjugated secondary antibody and detection with ECL. The graph represents the mean and range of two values: α5β1-integrin binding to peptide 240-248 (▪) or SuPAR (▴); α3β1-integrin binding to peptide 240-248 () and SuPAR (x). FIG. 1F shows that single amino acid substitution (S245A) in peptide 240-248 eliminates integrin binding. Peptide 240-248 (domain III), 17-24 (domain I), and the S245A mutant of peptide 240-248 were immobilized and tested for integrin binding as in FIG. 1A. FIGS. 1G-H show the crystal structure of human uPAR shown as a surface representation. The three individual domains of uPAR in FIG. 1G are indicated as follows: D1 is on the right side, DII is on the top of the model, and Diii is on the left of the model. In FIG. 1H, the order of the domains is inversed. The front view illustrates the deep central cavity that constitutes the uPA-binding site and the back view shows the partial exposure of the residues corresponding to the peptide 240-248 (indicated in dark gray). The location of the COOH-terminal in uPAR domain III harboring the GPI-anchor is indicated by the arrow (FIG. 1H). FIGS. 1G-H were created by the program PyMol (DeLano Scientific) using the coordinates 1YWH.

FIGS. 2A-B show solid phase dot-ELISA of suPAR. Mutant SuPAR and wild type suPAR at 0.5, 1.0, and 2.0 μg were dot-blotted onto the nitrocellulose membrane and incubated with 750 ng/ml purified human α5β1-integrin. Binding was detected as in FIG. 1A and the dots were scanned with NIH Image. Mean of the two-scanned value for each suPAR concentration are shown in the bottom panel. Mutation of residues in the 239-249 sequence reduced the integrin binding by ˜50 to 94%. suPAR^{E33A} (▪), suPAR^{wt} (▴), suPAR^{R239A} (♦), suPAR^{H249A} (x), suPAR^{Q248A} (∘), suPAR^{M246A} (), suPAR^{S245A} (□).

FIGS. 3A-C illustrate that SuPAR^{S245A} expressed in cells has reduced ability to activate α5β1-integrin. FIG. 3A shows FACS analysis of HUTS-4 antibody binding. HEK293 cells were transfected with pcDNA3.1 (vector), uPAR^{wt}, or uPAR^{S245A} and used 48 hrs later. Vector-transfected cells incubated with medium alone or with medium with 1 mM MnCl₂, followed by HUTS-4 and uPAR-transfected cells (uPAR^{wt} and uPAR^{S245A}) incubated with HUTS-4 or isotype matched Ig (IgG2b) were then incubated with secondary rabbit-anti mouse IgG coupled to Alexa 488. uPAR expression was detected using anti-uPAR antibody (R2). In the upper panels, the results are plotted as number of events vs. fluorescence intensity. The numbers indicate percent of total population above the median fluorescence intensity determined in vector-transfected cells. The lower panel relates to overlay of uPAR in uPAR^{wt} (black line) and uPAR^{S245A} (grey line) transfected cells. FIG. 3B shows cell-surface binding of fibronectin and fibronectin fibril formation. HEK293 cells were transiently transfected with uPAR^{wt} or uPAR^{S245A} expressing constructs and five hrs after transfection seeded on coverslips in medium with FBS depleted of fibronectin, with 30 μg/ml human fibronectin (FIG. 3B, left panels) or with 20 μg/ml of α5β1-integrin blocking antibody (FIG. 3B, middle panels). Cells in the right panels of FIG. 3B were incubated in medium depleted of plasminogen with 15 nM pro-uPA. Cell-bound fibronectin was detected by immunofluorescence 48 hrs after transfection. Nuclei were stained with DAPI. Bar=10μ. FIG. 3C shows deoxycholate (“DOC”)-insoluble fibronectin. HEK293 cells plated in 60 mm dishes were transfected and treated as in FIG. 3B, and the DOC-insoluble fibronectin was analyzed by 6% PAGE and Western blot under non-reducing conditions. The purified fibronectin band is ˜440 kDa. ERK shown as a loading control.

FIGS. 4A-C show that the uPAR^{wt} but not the uPAR^{S245A} mutant enhances adhesion of HEK293 cells to fibronectin. FIG. 4A shows that uPAR^{S245A} does not stimulate cell adhesion to fibronectin. HEK293 cells transiently transfected with pcDNA3.1, or expression constructs for uPAR^{wt} or uPAR^{S245A} were detached 40 hrs after transfection and 1.5×10⁵ cells were seeded on fibronectin (4 μg/ml in a 48-well plate) for 15 and 30 min at 37° C. Cell adhesion was quantified as described in the Examples. The results shown are mean (SD) of 2 individual experiments, 4 determinations for each cell type. Two additional experiments were performed with similar results. FIG. 4B shows the effect of uPA and anti-uPAR antibody (R2) on adhesion to fibronectin—HEK293 cells transiently transfected with expression constructs for uPAR^{wt} and uPAR^{S245A} as in FIG. 4A incubated in suspension without or with pro-uPA (10 nM). The cells were divided into 2 aliquots and one aliquot was incubated for 10 min with anti-uPAR(R²) antibody (10 μg/ml). Cells (1.2×10⁵) were seeded onto wells of a 48-well plate and the adhesion to fibronectin tested as in FIG. 4A. The results are mean (SD) of 6 determinations. Pro-uPA treatment of uPAR^{wt} cells increased adhesion significantly (p=0.00 by t-test) while the increase of adhesion in uPA-treated uPAR^{S245A} cells was not significant (p=0.14). The inset of FIG. 4B shows uPAR levels determined by Western blot. FIG. 4C shows the effect of peptides RAD, RGD, uPAR 17-24, and 240-248 on adhesion to fibronectin-HEK293 cells (transiently transfected to express uPAR^{wt} as in FIG. 4A) and T-HEp3 cells (endogenously expressing high levels of uPAR) incubated for 15 min in suspension with peptides RAD, RGD (500 μM each), 17-24, and 240-248 (20 and 200 μM for each). Cells were seeded onto 96-well plates and adhesion to fibronectin tested as in FIG. 4A. The results are mean (SD) of 3 determinations for each peptide. The experiment was repeated twice.

FIGS. 5A-B show the physical interaction between uPAR and α5β1-integrin. FIG. 5A shows the effect of S245A mutation on integrin/uPAR co-immunoprecipitation. D-HEp3 cells were transfected with vector (pcDNA3.1) alone or with constructs expressing uPAR^{wt} or uPAR^{S245A} and after 48 hrs the cells were surface biotinylated and lysed as described in the Examples. Half of each lysate (0.8 mg of protein) was immunoprecipitated with 5 μg monoclonal anti-uPAR(R3) antibody and half with 5 μg monoclonal anti-α5β1-integrin (HA5) antibody. Isotype-matched IgG served as negative control. The immunoprecipitates were separated on an SDS-PAGE and the bands were detected with streptavidin-HRP conjugate and ECL. The left single panel of FIG. 5A shows uPAR^{wt} transfected cell lysates protein (0.8 mg) immunoprecipitated with anti-α5β1-integrin and probed with a rabbit polyclonal anti-uPAR antibody. The numbers represent uPAR precipitated by anti-α5β1 antibody as percent of total uPAR. The experiment was repeated 5 times using the same and additional anti-integrin and anti-uPAR antibodies with essentially similar results. FIG. 5B is a graph showing the disruption of uPAR and α5β1-integrin complex with peptides 240-248 and S245A. T-HEp3 cells were surface biotinylated and lysed as described in the Examples. The lysate was precleared with isotype-matched IgG and aliquots containing 0.8 mg protein were incubated with peptides 240-248 and S245A (5 μM and 20 μM) or without peptides (Control, C) for 20 min at 4° C. followed by IP with 5 μg of monoclonal anti-α5β1-integrin (HAS) antibody for 90 min. The immunoprecipitates were analyzed as in FIG. 5A and scanned with NIH Image. The bars show mean (SD) of uPAR pulled down by anti-α5β1-integrin antibody as percent of control without peptide.

FIGS. 6A-E show ERK activation and growth in vivo. In FIG. 6A, HEK293 and D-Hep3 cells transfected with uPAR^{wt} produces stronger activation of ERK than cells transfected with uPAR^{S245A} or with vector (top and middle panels); HEK293, and D-HEp3 cells, transiently transfected with pcDNA3.1 vector, or with an expression construct for uPAR^{wt} or uPAR^{S245A} lysed 48 hrs later and analyzed by Western blotting for phospho-ERK (FIG. 6A, top panels) and ERK 1/2 (FIG. 6A, middle panels) with specific antibodies. The bands were scanned with NIH Image and plotted. The ratio of phospho-ERK to ERK was calculated and expressed as a fraction of the ratio of pcDNA3.1 transfected cells. In FIG. 6A (bottom panels), uPAR^{wt} and uPAR^{S245A} expression was determined by Western blotting. HEK293 cells do not express uPAR. In FIG. 6B, uPAR^{S245A} does not provide a proliferative stimulus in vivo. D-HEp3 cells transfected with vector, or uPAR^{wt} or uPAR^{S245A} were detached with EDTA 36 hrs after transfection and 7×10⁵ cells in 30 μl PBS were inoculated onto chorioallantoic membranes (“CAMs”) of 10-day old chick embryos. Each sample was inoculated onto 4 CAMs. The CAMs were excised 4 days later and the number of tumor cells was determined as in the Examples. The bars show mean and standard deviation. Paired t-test vector/uPAR^{S245A} p=0.76, vector/uPAR^{wt} p=0.0005, uPAR^{S245}/uPAR^{wt} p=0.0002. Inset: uPAR expression detected by Western blot with R2 antibody. In FIG. 6C, peptide 240-248, but not peptide 17-24, down regulates P-ERK level. T-HEp3 cells were serum-starved and treated in serum-free medium with 25 μM of peptides 240-248 and 17-24 for 45 min and 3 hrs. The cells were lysed and the lysates were analyzed for P-ERK (FIG. 6C, upper panel) and total ERK (FIG. 6C, lower panel) by Western blotting. FIG. 6D is a graph showing that S245A mutant peptide has no effect on ERK activation. T-HEp3 cells were treated with 5, 20, and 40 μM peptides 240-248 (), 17-24 (▪), and S245A (▾) for 1 hr. The cells were processed as in FIG. 6C, except that the bands were scanned by NIH Image the P-ERK/ERK ratio was calculated. The results show mean and SE of 3 determinations. The results show mean and SE of three determinations (FIG. 6D). FIG. 6E shows the effect of peptide 240-248 on ERK activation in T-ELK:GFP cells. Sub-confluent T-ELK:GFP cells grown in 48-well plate and serum-starved overnight were incubated with the uPAR peptides 240-248 at 5 and 25 μM and with 17-24 peptide at 25 μM for 40 hrs at 37° C. in serum-free medium. The cells were detached and analyzed for GFP in FACS Canto using FACSDiva software. The numbers represent GFP-positive cells as percent of total. The dose-response for peptide 240-248 and highest concentration of peptides 17-24 and S245A were repeated two additional times with similar results.

FIGS. 7A-B show the chemical structures of two compounds, MS0012479 and MS0019128.

FIG. 8 is a graph showing the experimental results of two compounds selected for activity as ERK inhibitors retested in 3 individual experiments.

FIG. 9 is a graph showing the results of a structure activity relationship analysis of activity for analogs of MS0012479 (FIG. 7A) and of MS0019128 (FIG. 7B).

FIG. 10 shows the chemical structure of the compound MS0124305.

FIG. 11 is a flow chart summarizing how compounds were selected pursuant to methods of the present invention.

FIG. 12 is a pair of graphs showing effect of compound MS0012479 on T-Hep3 tumor growth in vivo on chorioallantoic membrane.

### DETAILED DESCRIPTION OF THE INVENTION

One aspect of the present invention is directed to a method of inducing cancer cells into dormancy and treating cancer in a subject. This method involves administering to a subject an effective amount of a compound that disrupts interaction between urokinase plasminogen activator receptor and integrin, thereby inducing cancer cells into dormancy and treating cancer in the subject.

In a preferred embodiment, compounds that disrupt the interaction between urokinase plasminogen activator receptor and integrin include compounds having the following formula:

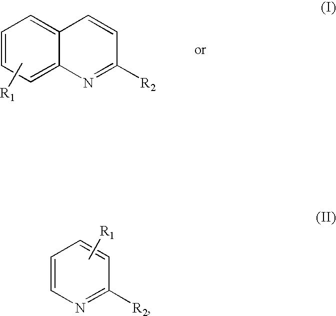

where:

R₂ is

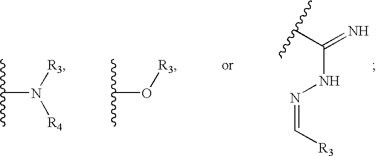

R₃ is

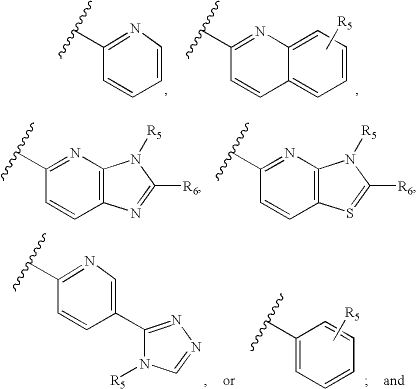

R₁, R₄, R₅, and R₆ are independently selected from the group consisting of hydrogen, —(CH₂)ₙOH, —(CH₂)ₙNH₂, —(CH₂)ₙNHCH₃, —(CH₂)ₙNHCH₂CH₃, —(CH₂)ₙCOOH, halo, lower alkyl, haloalkyl, —(CH₂)ₙ—SH, lower thioalkyl, —CN, lower alkoxy, and

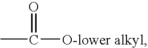

where n is an integer from 0 to 4, and pharmaceutically acceptable salts thereof.

As used herein, the term “lower alkyl” is defined as an alkyl group having 1 to 4 carbons that can be a straight or branched chain. Exemplary lower alkyl groups include, without limitation, methyl, ethyl, propyl, isopropyl, butyl, isobutyl, sec-butyl, and tent-butyl. The methyl group is preferred.

The term “halo” refers to F, Cl, Br, and I. Of these, F, Cl, and Br are preferred.

The term “lower thioalkyl” is defined as a lower alkyl group linked to a sulfur atom. Exemplary lower thialkyl groups include, without limitation, —SCH₃, —SCH₂CH₃, —S(CH₂)₂, CH₃, etc. Of these, —SCH₃ is preferred.

The term “haloalkyl” is defined as a lower alkyl group containing on e or more halo atoms. Exemplary haloalkyl groups include, without limitation, fluoroalkyl, bromoalkyl, chloroalkyl, difluoroalkyl, dibromoalkyl, dichloroalkyl, trifluoroalkyl, tribromoalkyl, and trichloroalkyl. Trifluoromethyl, trichloromethyl, and tribromomethyl are preferred.

The term “lower alkoxy” is defined as a lower alkyl group linked to an oxygen atom. Exemplary lower alkoxy groups include, without limitation, methoxy, ethoxy, and propoxy. Of these, methoxy and ethoxy are preferred.

For the substituents —(CH₂)ₙNH₂, —(CH₂)ₙNHCH₃, —(CH₂)ₙNHCH₂CH₃, —(CH₂)ₙOH, —(CH₂)ₙSH, and —(CH₂)ₙ—COOH, n is preferably 0 or 1. When n is 0, the substituent is the amine group, hydroxyl group, thiol group, or carboxylic acid group.

The compounds described herein can be made using known synthesis methods in the art and, in particular, guidance from methods described in Madrid et al., “Synthesis of Ring-Substituted 4-aminoquinolines and Evaluation of their Antimalarial Activities,” Biorg. Med. Chem. Lett. 15:1015-1018 (2005); Seeman, “The Woodward-Doering/Rabe-Kindler Total Synthesis of Quinine. Setting the Record Straight,” Angew. Chem. Int. Ed. 46:1378-1413 (2007); and Kumura et al., “Synthesis and Biological Activity of Fatty Acid Derivatives of Quinine,” Biosci. Biotechnol. Biochem. 69:2250-2253 (2005), which are hereby incorporated by reference in their entirety.

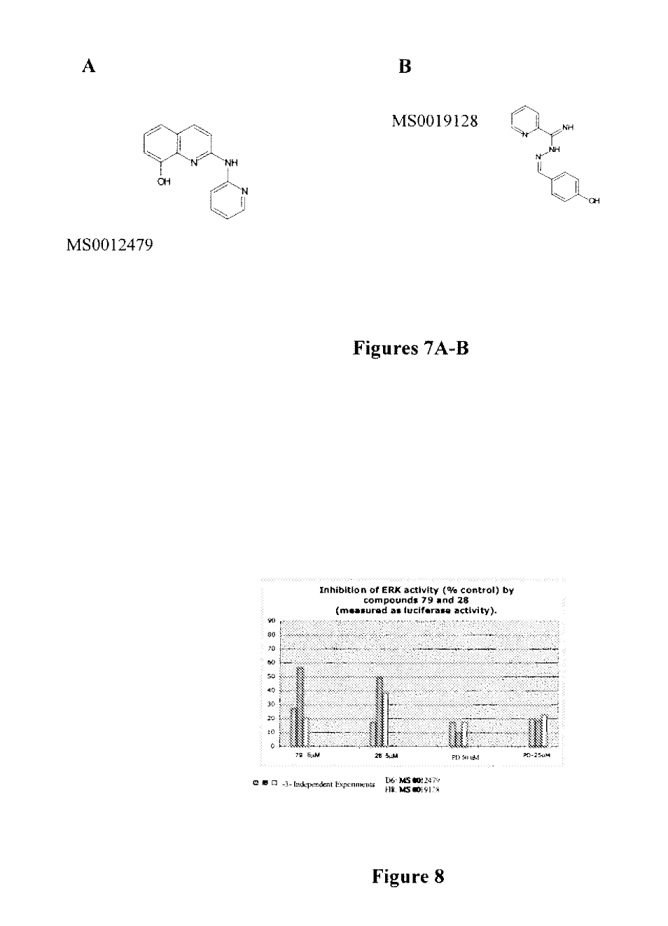

Particular compounds of Formula I suitable in carrying out this and other methods of the present invention include, without limitation, MS0012479 (FIG. 7A), MS0124305 (FIG. 10), MS0012476,

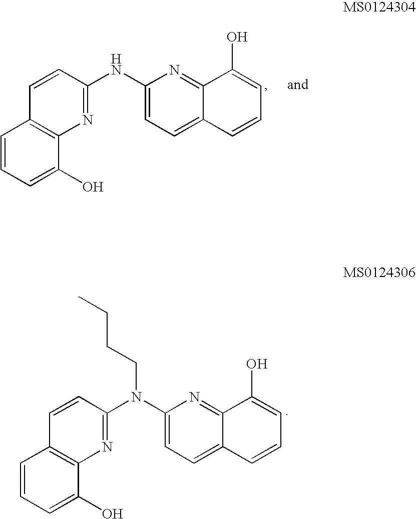

Particular compounds of Formula II suitable in carrying out this and other methods of the present invention include, without limitation, MS0019128 (FIG. 7B) and

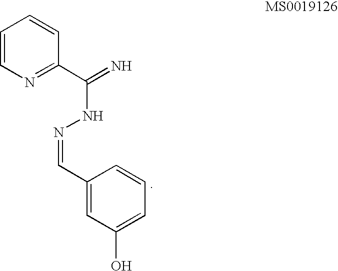

Pursuant to this method of the present invention, therapeutic agents are administered to a subject diagnosed with cancer, i.e., having established cancer in the subject, to inhibit the further growth or spread of the malignant cells, and/or to cause dormancy or death of the malignant cells. In particular, head and neck squamous cell carcinoma (HNSCC), breast cancer, ovarian cancer, prostate cancer, colon cancer, squamous carcinoma of the skin, glioblastoma, endometrial carcinoma, gastric cancer, pancreatic cancer, renal cell carcinoma, squamous cell lung cancer, and bladder cancer are amenable to the treatment in accordance with the method of the present invention. Treating cancer and inducing cancer cells into dormancy also encompasses treating a subject having premalignant conditions to stop the progression of, or cause regression of, the premalignant conditions. Examples of premalignant conditions include hyperplasia, dysplasia, and metaplasia.

In practicing the method of inducing cancer cells into dormancy and treating cancer in a subject of the present invention, the administering step is carried out by administering an agent directly to the tumor site, systemically, or both. The specific mode of administration may depend on the type of cancer being treated. Exemplary modes of administration include, without limitation, orally, topically, transdermally, parenterally, subcutaneously, intravenously, intramuscularly, intraperitoneally, by intranasal instillation, by intracavitary or intravesical instillation, intraocularly, intraarterially, intralesionally, by inhalation, or by application to mucous membranes. The agent of the present invention may be administered alone or with suitable pharmaceutical carriers, and can be in solid or liquid form, such as tablets, capsules, powders, solutions, suspensions, or emulsions.

The relative activity, potency, and specificity of the compound may be determined by a pharmacological study in animals, for example, according to the method of Nyberg et al., Psychopharmacology 119:345-348 (1995), which is hereby incorporated by reference in its entirety. Although the differential metabolism among patient populations can be determined by a clinical study in humans, less expensive and time-consuming substitutes are provided by the methods of Kerr et al., Biochem. Pharmacol. 47:1969-1979 (1994) and Karam et al., Drug Metab. Discov. 24:1081-1087 (1996), which are hereby incorporated by reference in their entirety. The potential for drug-drug interactions may be assessed clinically according to the methods of Leach et al., Epilepsia 37:1100-1106 (1996), which is hereby incorporated by reference in its entirety, or in vitro according to the methods of Kerr et al., Biochem. Pharmacol. 47:1969-1979 (1994) and Turner et al., Can. J. Physio. Pharmacol. 67:582-586 (1989), which are hereby incorporated by reference in their entirety.

The magnitude of the agent, or a pharmaceutically acceptable salt or derivative thereof, will vary with the nature and severity of the condition to be treated and the route of administration. The dose, and perhaps the dose frequency, will also vary according to the age, body weight, and response of the individual subject. The total daily dose of compounds of agents may be administered in single or divided doses.

The compounds should be administered in an effective amount. Exemplary doses of compounds for oral administration typically range from about 1 mg per unit dose to 2,000 mg per unit dose and more typically from about 10 mg per unit dose to 500 mg per unit dose. Preferably, the dosage is in the range of 1.0 to 200 mg/kg/day and the preferred dosage range is 1.0 to 50 mg/kg/day.

It is further recommended that children, subjects over 65 years old, and those with impaired renal or hepatic function, initially receive low doses and that the dosage be titrated based on individual responses and blood levels. It may be necessary to use dosages outside these ranges in some cases, as will be apparent to those of ordinary skill in the art. Further, it is noted that the clinician or treating physician knows how and when to interrupt, adjust, or terminate therapy in conjunction with and individual subject's response.

Pharmaceutical compositions of the present invention may include a pharmaceutically acceptable carrier, and optionally, other therapeutic ingredients or excipients.

The term “pharmaceutically acceptable salt thereof” refers to salts prepared from pharmaceutically acceptable, non-toxic acids including inorganic acids and organic acids, such as, for example, acetic acid, benzenesulfonic (besylate) acid, benzoic acid, camphorsulfonic acid, citric acid, ethenesulfonic acid, fumaric acid, gluconic acid, glutamic acid, hydrobromic acid, hydrochloric acid, isethionic acid, lactic acid, maleic acid, malic acid, mandelic acid, methanesulfonic acid, mucic acid, nitric acid, pamoic acid, pantothenic acid, phosphoric acid, succinic acid, sulfuric acid, tartaric acid, and p-toluenesulfonic acid.

The pharmaceutical compositions may be conveniently presented in unit dosage form, and may be prepared by any of the methods well known in the art of pharmacy. Preferred unit dosage formulations are those containing an effective dose, or an appropriate fraction thereof, of the active ingredients.

The compositions of the present invention may include a pharmaceutically acceptable carrier. The carrier may take a wide variety of forms, depending on its desired administration, for example, oral or parenteral (including intravenous). In preparing the composition for oral dosage form, any of the usual pharmaceutical media may be employed, such as, water, glycols, oils, alcohols, flavoring agents, preservatives, and coloring agents in the case of oral liquid preparation, including suspension, elixirs and solutions. Carriers such as starches, sugars, microcrystalline cellulose, diluents, granulating agents, lubricants, binders and disintegrating agents may be used in the case of oral solid preparations such as powders, capsules and caplets, with the solid oral preparation being preferred over the liquid preparations. Preferred solid oral preparations are tablets or capsules, because of their ease of administration. If desired, tablets may be coated by a standard aqueous or nonaqueous technique. Oral and parenteral sustained release dosage forms may also be used.

Oral syrups, as well as other oral liquid formulations, are well known to those skilled in the art, and general methods for preparing them are found in any standard pharmacy school textbook. For example, chapter 86, of the 19th Edition of Remington: The Science and Practice of Pharmacy, entitled “Solutions, Emulsions, Suspensions and Extracts,” describes in complete detail the preparation of syrups (pages 1503-1505, which are hereby incorporated by reference in their entirety) and other oral liquids.

Similarly, sustained release formulations are well known in the art, and Chapter 94 of the same reference, entitled “Sustained-Release Drug Delivery Systems,” describes the more common types of oral and parenteral sustained-release dosage forms (pages 1660-1675, which are hereby incorporated by reference in their entirety). Because they reduce peak plasma concentrations, as compared to conventional oral dosage forms, controlled release dosage forms are particularly useful for providing therapeutic plasma concentrations while avoiding the side effects associated with high peak plasma concentrations that occur with conventional dosage forms.

The solid unit dosage forms can be of the conventional type. The solid form can be a capsule, such as an ordinary gelatin type containing the betulinol derivative and a carrier, for example, lubricants and inert fillers, such as lactose, sucrose, or cornstarch. In another embodiment, agents can be tableted with conventional tablet bases, such as lactose, sucrose, or cornstarch, in combination with binders, like acacia, cornstarch, or gelatin, disintegrating agents, such as cornstarch, potato starch, or alginic acid, and lubricants, like stearic acid or magnesium stearate.

The pharmaceutical compositions may also be administered in injectable dosages by solution or suspension of these materials in a physiologically acceptable diluent with a pharmaceutical carrier. Such carriers include sterile liquids, such as water and oils, with or without the addition of a surfactants, adjuvants, excipients, or stabilizers. Illustrative oils are those of petroleum, animal, vegetable, or synthetic origin, for example, peanut oil, soybean oil, or mineral oil. In general, water, saline, aqueous dextrose and related sugar solutions, and glycols, such as propylene glycol or polyethylene glycol, are preferred liquid carriers, particularly for injectable solutions.

For use as aerosols, the pharmaceutical compositions in solution or suspension may be packaged in a pressurized aerosol container together with suitable propellants, for example, hydrocarbon propellants like propane, butane, or isobutane, and with conventional adjuvants. The pharmaceutical compositions may also be administered in a non-pressurized form, such as in a nebulizer or atomizer.

Preferred subjects in accordance with the methods of the present invention include, without limitation, any mammal, preferably a human.

Another aspect of the present invention is directed to a method of disrupting interaction between integrin and urokinase plasminogen activator receptor on cancer cells. This method involves contacting cancer cells with an effective amount of a compound as described above.

In carrying out this method of the present invention, contacting cancer cells may be carried in vitro or in vivo.

By “disrupting interaction” it is meant that the interaction necessary to growth and development of cancer cells is interfered with, so that cancer cells are unable to metastasize and to grow.

A further aspect of the present invention is directed to a method of screening for compounds effective in inducing tumor dormancy and treating cancer in a subject. This method involves providing one or more candidate compounds. The one or more candidate compounds are contacted with urokinase plasminogen activator receptor under conditions effective to disrupt interaction between integrin and the urokinase plasminogen activator receptor. Candidate compounds which disrupt interaction between integrin and urokinase plasminogen activator receptor are identified as compounds potentially effective in inducing tumor dormancy and treating cancer in a subject.

In carrying out the method of screening for compounds of the present invention, a cell is provided which expresses a urokinase plasminogen activator receptor (uPAR). To this end, a nucleic acid molecule encoding a uPAR polypeptide or protein can be introduced into an expression system of choice using conventional recombinant technology. Generally, this involves inserting the nucleic acid molecule into an expression system to which the molecule is heterologous (i.e., not normally present). The introduction of a particular foreign or native gene into a mammalian host is facilitated by first introducing the gene sequence into a suitable nucleic acid vector. “Vector” is used herein to mean any genetic element, such as a plasmid, phage, transposon, cosmid, chromosome, virus, virion, etc., which is capable of replication when associated with the proper control elements and which is capable of transferring gene sequences between cells. Thus, the term includes cloning and expression vectors, as well as viral vectors. The heterologous nucleic acid molecule is inserted into the expression system or vector in proper sense (5′→3′) orientation and correct reading frame. The vector contains the necessary elements for the transcription and translation of the inserted uPAR sequence.

U.S. Pat. No. 4,237,224 to Cohen and Boyer, which is hereby incorporated by reference in its entirety, describes the production of expression systems in the form of recombinant plasmids using restriction enzyme cleavage and ligation with DNA ligase. These recombinant plasmids are then introduced by means of transformation and replicated in unicellular cultures including prokaryotic organisms and eukaryotic cells grown in tissue culture.

Recombinant genes may also be introduced into viruses, including vaccinia virus, adenovirus, and retroviruses, including lentivirus. Recombinant viruses can be generated by transfection of plasmids into cells infected with virus.

Suitable vectors include, but are not limited to, the following viral vectors such as lambda vector system gt11, gt WES.tB, Charon 4, and plasmid vectors such as pBR322, pBR325, pACYC177, pACYC184, pUC8, pUC9, pUC18, pUC19, pLG339, pR290, pKC37, pKC101, SV 40, pBluescript II SK +/− or KS +/−(see “Stratagene Cloning Systems” Catalog (1993) from Stratagene, La Jolla, Calif., which is hereby incorporated by reference in its entirety), pQE, pIH821, pGEX, pET series (see F. W. Studier et. al., “Use of T7 RNA Polymerase to Direct Expression of Cloned Genes,” Gene Expression Technology Vol. 185 (1990), which is hereby incorporated by reference in its entirety), and any derivatives thereof. Recombinant molecules can be introduced into cells via transformation, particularly transduction, conjugation, mobilization, or electroporation. The DNA sequences are cloned into the vector using standard cloning procedures in the art, as described by Sambrook et al., Molecular Cloning: A Laboratory Manual, Cold Springs Laboratory, Cold Springs Harbor, N.Y. (1989), which is hereby incorporated by reference in its entirety.

A variety of host-vector systems may be utilized to express the uPAR-encoding sequence in a cell. Primarily, the vector system must be compatible with the host cell used. Host-vector systems include but are not limited to the following: bacteria transformed with bacteriophage DNA, plasmid DNA, or cosmid DNA; microorganisms such as yeast containing yeast vectors; mammalian cell systems infected with virus (e.g., vaccinia virus, adenovirus, etc.); insect cell systems infected with virus (e.g., baculovirus); and plant cells infected by bacteria. The expression elements of these vectors vary in their strength and specificities. Depending upon the host-vector system utilized, any one of a number of suitable transcription and translation elements can be used.

Different genetic signals and processing events control many levels of gene expression (e.g., DNA transcription and messenger RNA (“mRNA”) translation).

Transcription of DNA is dependent upon the presence of a promoter which is a DNA sequence that directs the binding of RNA polymerase and thereby promotes mRNA synthesis. The DNA sequences of eukaryotic promoters differ from those of prokaryotic promoters. Furthermore, eukaryotic promoters and accompanying genetic signals may not be recognized in or may not function in a prokaryotic system and, further, prokaryotic promoters are not recognized and do not function in eukaryotic cells.

Similarly, translation of mRNA in prokaryotes depends upon the presence of the proper prokaryotic signals which differ from those of eukaryotes. Efficient translation of mRNA in prokaryotes requires a ribosome binding site called the Shine-Dalgarno (“SD”) sequence on the mRNA. This sequence is a short nucleotide sequence of mRNA that is located before the start codon, usually AUG, which encodes the amino-terminal methionine of the protein. The SD sequences are complementary to the 3′-end of the 16S rRNA (ribosomal RNA) and probably promote binding of mRNA to ribosomes by duplexing with the rRNA to allow correct positioning of the ribosome. For a review on maximizing gene expression see Roberts and Lauer, Methods in Enzymology, 68:473 (1979), which is hereby incorporated by reference in its entirety.

Promoters vary in their “strength” (i.e., their ability to promote transcription). For the purposes of expressing a cloned gene, it is desirable to use strong promoters in order to obtain a high level of transcription and, hence, expression of the gene. Depending upon the host cell system utilized, any one of a number of suitable promoters may be used. For instance, when cloning in E. coli, its bacteriophages, or plasmids, promoters such as the T7 phage promoter, lac promoter, trp promoter, recA promoter, ribosomal RNA promoter, the P_{R} and P_{L} promoters of coliphage lambda and others, including but not limited to, lacUV 5, ompF, bla, lpp, and the like, may be used to direct high levels of transcription of adjacent DNA segments. Additionally, a hybrid trp-lacUV5 (tac) promoter or other E. coli promoters produced by recombinant DNA or other synthetic DNA techniques may be used to provide for transcription of the inserted gene.

Bacterial host cell strains and expression vectors may be chosen which inhibit the action of the promoter unless specifically induced. In certain operons, the addition of specific inducers is necessary for efficient transcription of the inserted DNA. For example, the lac operon is induced by the addition of lactose or IPTG (isopropylthio-beta-D-galactoside). A variety of other operons, such as trp, pro, etc., are under different controls.

Specific initiation signals are also required for efficient gene transcription and translation in prokaryotic cells. These transcription and translation initiation signals may vary in “strength” as measured by the quantity of gene specific messenger RNA and protein synthesized, respectively. The DNA expression vector, which contains a promoter, may also contain any combination of various “strong” transcription and/or translation initiation signals. For instance, efficient translation in E. coli requires a SD sequence about 7-9 bases 5′ to the initiation codon (ATG) to provide a ribosome binding site. Thus, any SD-ATG combination that can be utilized by host cell ribosomes may be employed. Such combinations include but are not limited to the SD-ATG combination from the cro gene or the N gene of coliphage lambda, or from the E. coli tryptophan E, D, C, B, or A genes. Additionally, any SD-ATG combination produced by recombinant DNA or other techniques involving incorporation of synthetic nucleotides may be used.

Depending on the vector system and host utilized, any number of suitable transcription and/or translation elements, including constitutive, inducible, and repressible promoters, as well as minimal 5′ promoter elements may be used.

The uPAR protein-encoding nucleic acid, a promoter molecule of choice, a suitable 3′ regulatory region and, if desired, a reporter gene, are incorporated into a vector-expression system of choice to prepare a nucleic acid construct using standard cloning procedures known in the art, such as described by Sambrook et al., Molecular Cloning: A Laboratory Manual, Third Edition, Cold Spring Harbor Cold Spring Harbor Laboratory Press, New York (2001), which is hereby incorporated by reference in its entirety.

The nucleic acid molecule encoding a uPAR is inserted into a vector in the sense (i.e., 5′→3′) direction, such that the open reading frame is properly oriented for the expression of the encoded uPAR under the control of a promoter of choice. Single or multiple nucleic acids may be ligated into an appropriate vector in this way, under the control of a suitable promoter, to prepare a nucleic acid construct.

Once the isolated nucleic acid molecule has been cloned into an expression system, it is ready to be incorporated into a host cell. Recombinant molecules can be introduced into cells via transformation, particularly transduction, conjugation, lipofection, protoplast fusion, mobilization, particle bombardment, or electroporation. The DNA sequences are cloned into the host cell using standard cloning procedures known in the art, as described by Sambrook et al., Molecular Cloning: A Laboratory Manual, Second Edition, Cold Springs Laboratory, Cold Springs Harbor, N.Y. (1989), which is hereby incorporated by reference in its entirety. Suitable hosts include, but are not limited to, bacteria, virus, yeast, fungi, mammalian cells, insect cells, plant cells, and the like.

Typically, an antibiotic or other compound useful for selective growth of the transformed cells only is added as a supplement to the media. The compound to be used will be dictated by the selectable marker element present in the plasmid with which the host cell was transformed. Suitable genes are those which confer resistance to gentamycin, G418, hygromycin, puromycin, streptomycin, spectinomycin, tetracycline, chloramphenicol, and the like. Similarly, “reporter genes,” which encode enzymes providing for production of an identifiable compound, or other markers which indicate relevant information regarding the outcome of gene delivery, are suitable. For example, various luminescent or phosphorescent reporter genes are also appropriate, such that the presence of the heterologous gene may be ascertained visually.

According to this method of the present invention, “contacting” can be carried out as desired, including, but not limited to, physically contacting cells in culture with compounds to be selected in a suitable growth medium. Alternatively, mice, rats or other mammals are injected with compounds to be selected. Contacting can also be carried by computer simulation.

In a preferred embodiment of this method of the present invention, “contacting” involves contacting the one or more candidate compounds with a fragment of urokinase plasminogen activator receptor having the sequence of SEQ ID NO:9.

These aspects of the present invention are further illustrated by the Examples below.

### EXAMPLES

The following Examples are provided to illustrate embodiments of the present invention, but they are by no means intended to limit its scope.

### Example 1

### Reagents and Antibodies

Bovine serum albumin, dimethylsulfoxide (DMSO), Triton X-100, sodium orthovanadate, sodium fluoride, and human fibronectin were purchased from Sigma Chemical Co. (St. Louis, Mo.). Aprotinin and trypsin were from ICN Biomedicals, Inc. (Aurora, Ohio). DMEM, OPTI-MEM medium, glutamine, antibiotics, and lipofectin were from LifeTechnologies, Inc. (Grand Island, N.Y.). Fetal bovine serum was from JRH Biosciences (Lenexa, Kans.), COFAL-negative embryonated eggs were from specific Pathogen-Free Avian Supply (North Franklin, Conn.), protein G-agarose beads were from Roche Molecular Systems Inc. (Branchburg, N.J.), polyvinylidene difluoride membranes and ECL detection reagents were from Amersham (Amersham Life Sciences, Little Chalfont, United Kingdom), and protran nitrocellulose 0.2 μM (Schleicher and Schuell) was from PerkinElmer Life Sciences (Boston, Mass.). The plasmid pIRES-EGFP was from Clontech (Paolo Alto, Calif.). Anti-phospho-ERK1/2 (anti-phospho-tyr-204, clone E4) was from Santa Cruz Biotechnology (Santa Cruz, Calif.) anti-ERK1/2 (clone MK12) was from Transduction Laboratories (Lexington, Ky.), and BIIG2 (α5β1-integrin function blocking antibody) was from Developmental Studies Hybridoma Bank, (Iowa City, Iowa.). Purified laminin, human integrin α5β1, α3β1, rabbit anti-laminin antibody, anti-α5β1 antibody (HA5), and rabbit anti-integrin α5, α3 polyclonal antibodies were from Chemicon International (Temecula, Calif.). Rabbit anti-uPAR polyclonal antibody was from American Diagnostica Inc. (Stamford, Conn.). One Shot INVαF′ competent cells and Alexa Fluor 488 F(ab′)₂ fragment of rabbit anti-mouse IgG (H+L) were from Molecular Probes, Invitrogen (Carlsbad, Calif.). QuickChange Site-directed mutagenesis kit was from Stratagene (LaJolla, Calif.). Anti-mouse IgG monoclonal antibody conjugated with horseradish peroxidase (HRP) was from Vector Laboratories (Burlingame, Calif.). Monoclonal anti-uPAR domain III (R2) and domain I (R3) antibodies were prepared as described previously (Ronne et al., FEBS Lett. 288:233-236 (1991), which is hereby incorporated by reference in its entirety). Synthetic, non-uPA binding peptides (Liang et al., J. Biochem. (Tokyo) 134:661-666 (2003), which is hereby incorporated by reference in its entirety) derived from the uPAR sequences were purchased from Biotrend (Köln, Germany) or from Genemed Synthesis, Inc. (San Francisco, Calif.) either biotinylated on N-termini or with unmodified termini and free thiol-groups in cysteines. Purified soluble uPAR^{wt} (residues 1-283) and single amino acid mutants were prepared as previously described (Gardsvoll et al., J. Biol. Chem. 274:37995-38003 (1999), which is hereby incorporated by reference in its entirety). Most uPAR mutants were produced in Drosophila Schneider cells, with the exception of E33A and Q248 that were expressed in Chinese Hamster Ovary (CHO) cells. Recombinant proteins expressed in both cell types are glycosylated on the same residues, but the proteins produced in CHO cells have a more complex glycosylation pattern (Gardsvoll et al., Protein Expr. Purif. 34:284-295 (2004); Ploug et al., J. Biol. Chem. 273:13933-13943 (1998), which are hereby incorporated by reference in their entirety). Fibronectin-depleted serum was prepared on a gelatin-Sepharose4B column as per manufacturer instruction, and plasminogen depleted serum was prepared by passing FBS twice through a lysine-Sepharose column.

### Example 2

### Solid Phase Dot-ELISA

Synthetic uPAR-derived peptides and mutant proteins were diluted to 0.5, 1, and 2 μg/100 μl of PBS. The samples were dot-blotted using Bio-Rad Dot-ELISA apparatus onto pre-wet Nitrocellulose (0.2 μm) membrane in duplicates under a low vacuum, blocked with 3% non-fat dry milk in Tris-buffered saline (TBS) (25 mM Tris, 150 mM NaCl, pH7.4) and 0.05% Tween 20 for 1 hr on a gently shaking platform, cut into strips that were placed on a Saran-wrapped ELISA plate, overlaid with 500 μl of purified human α5β1-integrin (750 ng/ml of PBS) and incubated for 90 min in a humidified chamber at RT. The strips, pinned to the bottom of a plastic box, were washed with TBS with 0.1% Tween 20, overlaid with 500 μl of rabbit anti-integrin α5 polyclonal antibody (1:650) for 1 hr at RT, washed with TBST twice, incubated with goat anti-rabbit IgG (H+L) HRP conjugated secondary antibody (1:2000) for 1 hr, washed 3 times with TBST, and developed with ECL. The dots were scanned with NIH image.

### Example 3

### Kinetics of Pro-uPA/uPAR Interaction Determined by Surface Plasmon Resonance

The interactions between immobilized pro-uPA (200-1000 RU) and purified uPAR, produced in S2-cells (Ploug et al., J. Biol. Chem. 273:13933-13943 (1998), which is hereby incorporated by reference in its entirety), were measured at 20° C. by a Biacore3000 using serial 2-fold dilutions ranging from 1-100 nM uPAR. Coupling of pro-uPA^{S356A} to the sensor chip by EDC/NHS and regeneration of the chip was performed essentially as previously described (Gardsvoll et al., Protein Expr. Pur 34:284-295 (2004), which is hereby incorporated by reference in its entirety). The rate constants kₒₙ and k_{off} were derived from these data by nonlinear least squares curve fitting using the BIAevaluation 4.1 software.

### Example 4

### Cell Lines

Tumorigenic human epidermoid carcinoma HEp3 (T-HEp3) cells were serially passaged on CAMs of chick embryos as described previously (Ossowski et al., Cell 35:611-619 (1983), which is hereby incorporated by reference in its entirety). To obtain dormant HEp3 (D-HEp3) cells, the T-HEp3 cells were passaged in culture for 120-170 passages as described (Ossowski et al., Cell 35:611-619 (1983), which is hereby incorporated by reference in its entirety). D-HEp3 cells express approximately 20% of uPAR found in T-HEp3 cells (Aguirre-Ghiso et al., J. Cell Biol. 147:89-104 (1999), which is hereby incorporated by reference in its entirety). HEK293 cells were obtained from ATCC (Manassas, Va.). Cells were cultured in DMEM with 10% heat-inactivated FBS, penicillin (500 units/ml), and streptomycin (200 μg/ml).

### Example 5

### Site-Directed Mutagenesis and Transfection

A construct expressing full-length uPAR-cDNA in the HindIII site of pcDNA3.1-Hyg from Invitrogen (Carlsbad, Calif.) was as described (Liu et al., Cancer Cell 1:445-457 (2002), which is hereby incorporated by reference in its entirety). Site-directed mutagenesis (S245 to alanine substitution in domain III of uPAR) was carried out according to manufacturer's instructions, Stratagene (La Jolla, Calif.) using pcDNA3.1-uPAR as template and the following two primers:

(SEQ ID NO: 1)
5′-GCTGTGCAACCGCCTCAATGTGCCAACATG-3′
(forward) and
(SEQ ID NO: 2)
5′-CATGTTGGCACATTGCGGCGGTTGAACAGC-3′
(reverse).

The mutation was validated by DNA sequencing using oligonucleotides

(SEQ ID NO: 3)
5′-GAGACTTTCCTCATTG-3′ (forward)
and
(SEQ ID NO: 4)
5′-AATGAGGAAAGTCTC-3′ (reverse)

for sequencing. D-HEp3 and HEK293 cells were transiently transfected with empty vector (pcDNA3.1), uPAR^{wt}, or uPAR^{S245A}, using Fugene reagent (3 μl Fugene/μg of DNA).

### Example 6

### Surface Biotinylation with Sulfo-NHS-Biotin and Cell Lysis

Forty eight hrs after transfection, subconfluent monolayers were washed three times with cold PBS, incubated with 5 ml of 0.5 mg/ml sulfo-NHS-biotin from Pierce (Rockford, Ill.) on ice for 20 min, washed twice with ice-cold PBS, scraped into 1 ml of pre-chilled PBS containing cocktail of protease inhibitors, and briefly spun at 4° C. The pellets were lysed for 30 min on ice in integrin lysis buffer (1% Triton X-100, 50 mM HEPES (pH7.5), 150 mM NaCl, 1 mM CaCl₂, 1 mM MgCl₂, 1 mM orthovanadate, 1 mM sodium flouride) containing a cocktail of proteinase inhibitors. The cell lysates were spun down at 14,000 rpm for 10 min at 4° C. The supernatants were collected and subjected to immunoprecipitation, SDS-PAGE, and biotinylated protein detection.

### Example 7

### Co-Immunoprecipitation of α5β1-Integrin and uPAR

Cell lysates (0.8 mg protein) were pre-cleared with protein G-agarose beads preincubated with isotype-matched IgG for 45 min at 4° C. on a rolling platform and the supernatants incubated for 3 hr at 4° C. with protein G-agarose beads to which 5 μg of anti-α5β1(HA5), R3 antibodies, or isotype-matched IgG was bound. The protein G-beads were washed twice with PBS and protease inhibitors and once with 0.1% Nonidet P-40 (NP-40), resuspended in 2×-Laemmli sample buffer, heated, separated on SDS-PAGE, transferred onto a PVDF membrane, probed either with rabbit anti-uPAR polyclonal antibody or streptavidin-HRP, washed, and developed using ECL and scanned with NIH image. To disrupt the preformed α5β1-integrin/uPAR complex, the pre-cleared cell lysates were incubated for 20 min at 4° C. with either the 240-248 peptide or the mutant S245A peptides (5 and 20 μM), followed by pull-down with anti-α5β1(HA5) antibody. The rest of the procedures were as in co-immunoprecipitation.

### Example 8

### Detection of ERK^{MAPK} Activity and the Effect of Peptides

To analyze ERK activity, D-HEp3 and HEK293 cells were transiently transfected with pcDNA3.1, uPAR^{wt} and uPAR^{S245A} and, 48 hrs after transfection, the cells were lysed in RIPA buffer (1% Triton X-100, 0.1% SDS, 10 mM Tris pH 8.0, 140 mM NaCl) for 30 min on ice, the lysates were centrifuged at 14,000 rpm for 10 min at 4° C. and the supernatants were analyzed by Western blotting using anti-P-ERK and anti-ERK antibodies. The level of uPAR-expression was also tested in the same lysates by Western blotting using R2 antibody. The bands produced by anti-P-ERK and ERK antibodies were scanned by NIH Image and the ratios of P-ERK to ERK were calculated.

To test the effect of uPAR/integrin inhibiting peptides on ERK activity, T-HEp3 cells transfected with two plasmids that report through GFP level on the state of ERK activation and designated T-ELK (Aguirre-Ghiso et al., Cancer Res. 63:1684-1695 (2003), which is hereby incorporated by reference in its entirety) were plated in 48-well plates, serum-starved overnight, and treated (in duplicates) with 5 and 25 μM of uPAR-derived synthetic peptides (240-248) or 25 μM (17-24) in DMEM for 42 hrs. Untreated cells served as positive control and cells not expressing GFP as negative control. The cells were detached and analyzed for GFP by FACS analysis using FACS Canto (Becton Dickinson, Calif.) and FACSDiva software. Alternatively, T-HEp3 cells, serum-starved overnight were incubated in serum-free medium with 5, 20, and 40 μM of uPAR-derived synthetic peptide 240-248, 17-24, or S245A for 1 hr or with 25 μM peptide 240-248 and 17-24 for 10 min, 45 min, and 3 hrs at 37° C. The cells were lysed in RIPA buffer and processed as described above. Concentration dependence of P-ERK inhibition was analyzed by scanning P-ERK and ERK bands with NIH-Image and expressing their ratios as % of the ratio in untreated control (T-HEp3) cells.

### Example 9

### Adhesion Assay

HEK293 cells were transiently transfected with pcDNA3.1, uPAR^{wt}, or uPAR^{S245A}. After 40 hrs the cells were detached with 4 mM EDTA, suspended in DMEM, inoculated (1.5×10⁴ per well) in a 48-well plate, pre-coated overnight at 4° C. with 4 μg/ml fibronectin, and blocked with 0.1% BSA for one hr at 37° C. Following 15 and 30 min incubation at 37° C., the cells were washed twice with PBS with CaCl₂ and MgCl₂, fixed with 1% glutaraldehyde, stained with 1% crystal violet for 10 min, washed, dried, destained with 10% methanol and 5% acetic acid, and the OD of the extracted dye was measured in triplicate using the ELISA microplate-reader ELX800 from Bio-Tek Instruments. Inc. (Shelton, Conn.) at 570 nm. To analyze the uPA induced adhesion to fibronectin, uPAR^{wt} and uPAR^{S245A} transfected HEK293 cells were incubated prior to adhesion assay in suspension in DMEM with uPA (10 nM) either in the presence or absence of anti-uPAR antibody R2 (10 μg/ml) for 10 min, washed with DMEM, and plated as above. To test the effect of peptides on adhesion to fibronectin, T-HEp3 and HEK293 cells (the latter transiently transfected with uPAR^{wt}) were incubated with DMEM, RGD, RAD peptide (500 μM) (as a positive and negative control of the assay), 240-248 peptide, or 17-24 peptide (20 and 200 μM) for 15 min at RT, and plated as above.

### Example 10

### Detection of Active-β1 Integrin by FACS Analysis

HEK293 cells were transiently transfected with pcDNA3.1, uPAR^{wt}, and uPAR^{S245}. After 48 hrs the cells were detached with 2 mM EDTA in PBS, resuspended in RPMI and aprotinin (20 μg/ml) at 5×10⁵ cells/100 μl. Vector transfected cells were incubated with or without MnCl₂, followed by HUTS-4 antibody (1.0 μg). uPAR^{wt} and uPAR^{S245A} transfected cells were incubated with HUTS-4 (1.0 μg) or isotype matched IgG2b, or R² (2.0 μg), or isotype matched IgG1, at 37° C. for 20 min, washed, and incubated with rabbit anti-mouse Alexa 488-coupled IgG (1:400) at 4° C. for 25 min. Finally, cells were washed and suspended in 400 μl of FACS buffer and analyzed in FACS Canto (Becton Dickinson, Calif.) using FACSDiva software. Cells incubated with the isotype-matched Ig were used to gate the HUTS-4 and R2, respectively.

### Example 11

### Detection of Cell-Associated Fibronectin by Immunofluorescence Microscopy

HEK293 cells were transiently transfected with uPAR^{wt} and uPAR^{S245A} and plated on coverslips 5 hrs after transfection. After 24 hrs in serum containing medium, the medium was replaced with medium with 10% fibronectin-depleted serum supplemented with human fibronectin (30 μg/ml) with or without 20 μg/ml of α5β1-blocking antibody (BIIG2). In another set, pro-uPA (15 nM) was added to medium with FBS from which plasminogen was removed and human fibronectin (30 μg/ml) was added. Forty-eight hrs after transfection the cells were stained with rabbit anti-human fibronectin antibody (Sigma), followed by goat anti-rabbit antibody coupled to Alexa 488, and the nuclei were stained with DAPI. The images were observed in a fluorescent Nikon Eclipse E600 microscope and photographed with SPOT-RT™ camera from Spot Diagnostic Instruments (Sterling Height, Mich.).

### Example 12

### Deoxycholate Insoluble Fibronectin

The methods used to extract fibronectin were as previously described (Aguirre-Ghiso et al., Mol. Biol. Cell 12:863-879 (2001), which is hereby incorporated by reference in its entirety), except that no β-mercaptoethanol was added to the sample buffer.

### Example 13

### Growth of uPAR^{wt} and uPAR^{S245A} Transfected D-HEp3 Cells In Vivo (CAM)

Semi-confluent D-HEp3 cells were transfected with vector alone or with uPAR^{wt} or uPAR^{S245A} mutant-expressing plasmids and 36 hrs later the cells were detached with EDTA, counted, and inoculated at 7×10⁵ cells per CAM on 4 eggs each, as previously described (Aguirre-Ghiso et al., Mol. Biol. Cell 12:863-879 (2001), which is hereby incorporated by reference in its entirety). One dish of each transfectant was lysed and used to determine uPAR expression by Western blotting using R2 antibody. Four days after inoculation the CAMs were excised, dissociated into single cells with collagenase (Aguirre-Ghiso et al., Mol. Biol. Cell 12:863-879 (2001), which is hereby incorporated by reference in its entirety), and the tumor cells, which are larger than the chick embryo cells, were counted. It was previously established that only 50% of the cells are recovered from the CAM 24 hrs after inoculation.

### Example 14

### A Synthetic uPAR-Derived Peptide Binds the α5β1-Integrin

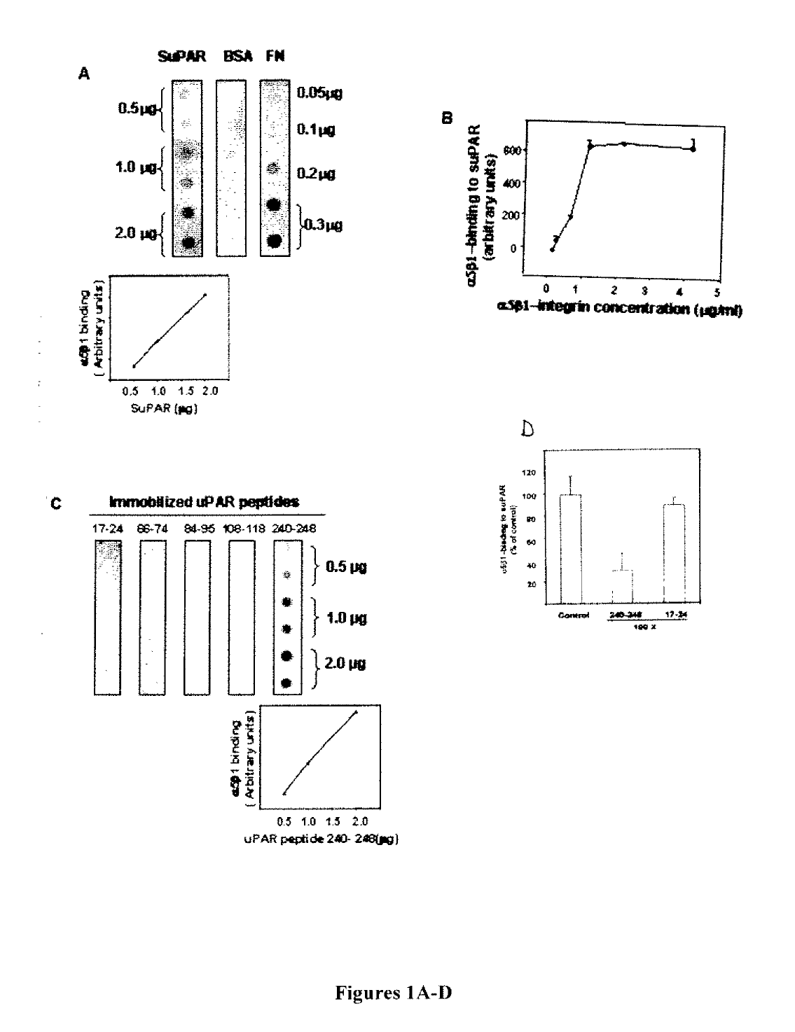

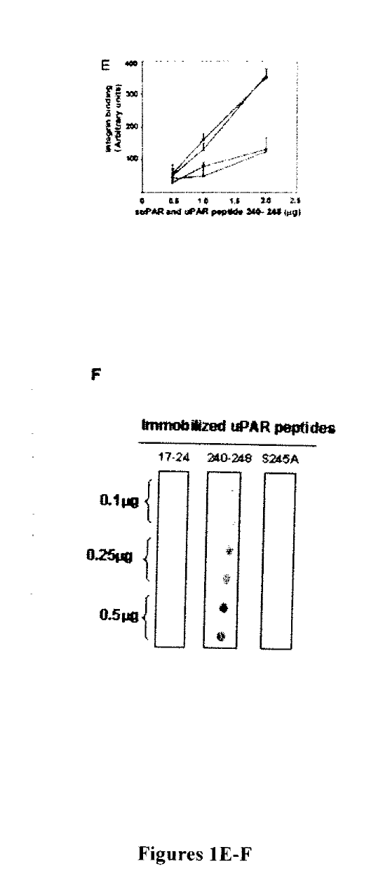

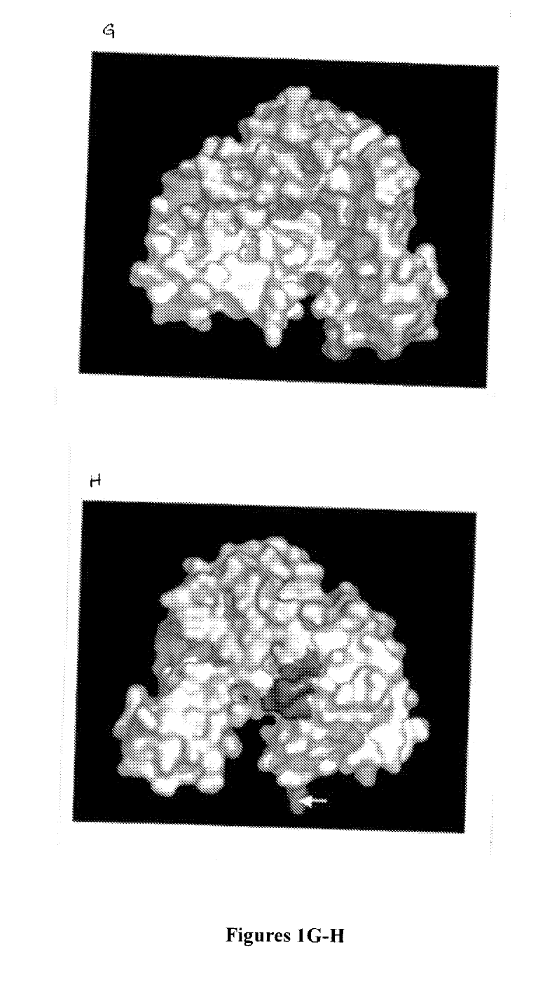

To establish parameters for peptide testing it was first examined whether the two full-length purified proteins, uPAR and α5β1-integrin, interact directly in a modified solid-phase Dot-ELISA protocol. Increasing concentrations of suPAR were immobilized on a nitrocellulose membrane and incubated with a constant amount of purified α5β1-integrin. The results (FIG. 1A) show a dose-dependent increase in band intensity of the bound integrin. As expected, the integrin bound to immobilized fibronectin while there was only background binding to BSA (FIG. 1A). A control in which the incubation step with a5131 was omitted yielded no detectable binding to either of the immobilized ligands. Binding of increasing concentrations of α5β1-integrin to a constant amount of uPAR reached saturation between 1.0 and 2.0 μg/ml of integrin (FIG. 1B).

To asses which part of the suPAR protein might be involved in the integrin binding, a collection of synthetic peptides corresponding to regions in human uPAR was used, which previously were tested for binding to uPAR and vitronectin (i.e., amino acid 17-24: CALGQDLC (SEQ ID NO:5); 66-74:LTEVVCGLD (SEQ ID NO:6) of domain I; 84-95: AVTYSRSRYLEC (SEQ ID NO:7); 108-118: GRHQSLQCRSP (SEQ ID NO:8) of domain II; and 240-248: GCATASMCQ (SEQ ID NO:9) of domain III) (Liang et al., J. Biochem. (Tokyo) 134:661-666 (2003), which is hereby incorporated by reference in its entirety). This peptide ensemble was screened individually for their integrin binding capacity. It was found that only one of the peptides tested, peptide 240-248 (SEQ ID NO:9), bound α5β1-integrin in a dose-dependent fashion (FIG. 1C). The remaining tested peptides showed no binding (FIG. 1C). To test whether peptide 240-248 can compete for integrin binding to suPAR, the purified α5β1-integrin was pre-incubated with excess of peptide prior to analysis in a Dot-ELISA. As shown in FIG. 1D, this treatment reduced the integrin binding to immobilized uPAR by more than 70%, while peptide in domain I of uPAR (residue 17-24) had only a negligible effect, suggesting that peptide 240-248 represents the integrin interacting site on suPAR.

To further probe the specificity of the newly identified interaction, another integrin of the β1-family, the α3β1, was tested. A set of control experiments revealed a similar band of intensity for α3β1-integrin bound to immobilized laminin-5 and for α5β1 bound to immobilized fibronectin when tested with antibodies titrated to produce bands of equal intensity on membrane-immobilized α3β1 or α5β1 integrins. It was found that, compared to α5β1-integrin, the purified α3β1-integrin produced dots of much lesser intensity when incubated with increasing concentrations of suPAR or peptide 240-248 (FIG. 1E).

Sequence alignment revealed that the integrin binding synthetic peptide, representing residues 240-248 in human uPAR, contains 4 positions that are conserved among human, horse, bovine, mouse, and rat uPAR (i.e., G240, C241, S245, and C247). A synthetic peptide with both cysteines replaced by alanines exhibited a reduction in uPAR binding activity, but even a more pronounced reduction was found when a single amino acid (S245) was replaced by alanine (FIG. 1F). The difference in integrin binding between peptide 240-248 and S245A shown in FIG. 1F was not due to unequal immobilization of the peptides to the membrane, which bound with the same efficiency as determined using biotinylated peptides. Precaution was taken to assure that the peptides remain in linear form by using only freshly solubilized aliquots and carrying out the experiments at room temperature to slow down the oxidation reaction. Importantly, as evident from the crystal structure of uPAR (Llinas et al., EMBO J. 24:1655-1663 (2005); Huai et al., Science 311:656-9 (2006), which are hereby incorporated by reference in their entirety) and the consensus for the Ly-6/uPAR/alpha-neurotoxin (Ploug et al., Biochemistry 33:8991-8997 (1994), which is hereby incorporated by reference in its entirety), these two cysteines do not form a disulfide bond in the natural uPAR protein. The integrin-binding peptide sequence identified is partly confined to the outer surface of the newly solved crystal structure of uPAR (FIGS. 1G-H). In particular, S245 and Q248 occupy a position that is distant from the uPA-binding cavity (Llinas et al., EMBO J. 24:1655-1663 (2005), which is hereby incorporated by reference in its entirety).

### Example 15

### The Effect of S245A Mutation in the uPAR Protein on Integrin Binding

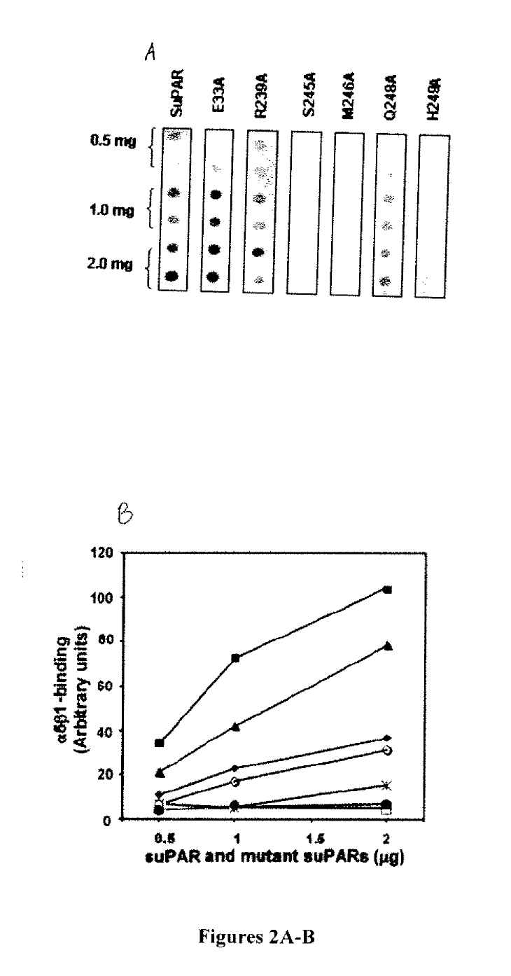

Impairment of integrin binding to these particular suPAR mutants were not the results of a gross misfolding of the proteins, because the rate constants determined by plasmon resonance using immobilized pro-uPA and purified wt or mutant uPAR (Table 1) were very similar. A single site mutant (L66A), previously shown to affect the uPA-uPAR interaction (Gardsvoll et al., Protein Expr. Pur 34:284-295 (2004), which is hereby incorporated by reference in its entirety) and used here as a control, showed a >5-fold increase in the k_{off} rate compared to uPAR^{wt} and a corresponding change in free energy of ΔΔG 1.35 kcal/mol. In contrast, the single site mutant that did not bind integrin (S245A, FIG. 2A) had only minimal change in k_{off} and free energy as compared to uPAR^{wt} (Table 1). Moreover, several monoclonal anti-uPAR antibodies recognizing conformational-dependent epitopes on uPAR bound equally well to the wt and S245A mutant uPAR. Thus, the identification of the sequence, which showed integrin-binding activity in vitro, permitted proceeding with biochemical and biological experiments in cells and in vivo. The functional role of the S245A mutation was further investigated.

TABLE 1
Rate Constants for the Interaction between Immobilized Pro-uPA
and uPAR as Measured by Surface Plasmon Resonance.

| uPAR | kₒₙ | k_{off} | K_{d} | ΔΔG |
|---|---|---|---|---|
| mutants | 10⁵ m⁻¹ S⁻¹ | 10⁻⁴ S⁻¹ | nm | kcal/mol |
| wt | 3.98 ± 0.98 | 1.77 ± 0.31 | 0.46 ± 0.11 | |
| E33A | 2.52 | 2.19 | 0.87 | 0.37 |
| L66A | 2.26 | 10.6 | 4.70 | 1.35 |
| R239A | 2.33 | 1.48 | 0.64 | 0.19 |
| S245A | 5.28 | 2.14 | 0.41 | −0.08 |
| H249A | 3.84 | 1.92 | 0.50 | 0.05 |

Shown are the rate constants derived for the interaction between immobilized pro-uPA^{S356A} and soluble uPAR (residues 1-283) as measured by surface Plasmon resonance at 20° C. A single site uPAR mutant (L66A) known to affect this interaction is included as a control (Liang et al., J. Biochem. (Tokyo) 134: 661-666 (2003), which is hereby incorporated by reference in its entirety). Values for uPAR wt are from >20 separate experiments. The K_{d} was calculated from the means of the corresponding rate constants (K_{d} = k_{off}/kₒₙ), and the change in Gibbs free energy was calculated as ΔΔG = RT ln[K_{d(mut)}/K_{d(wt)}].

### Example 16

### The GPI-Anchored uPAR^{S245A} has Impaired Ability to Activate α5β1-Integrin

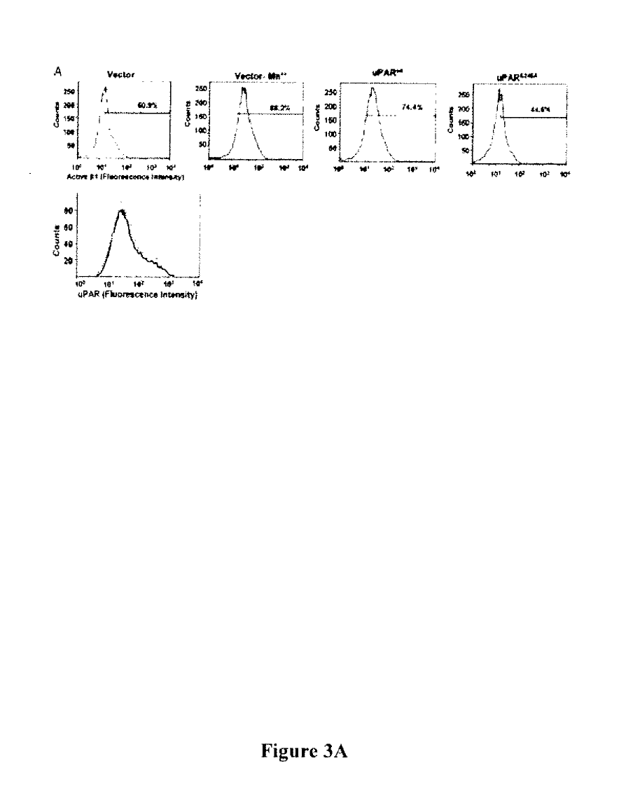

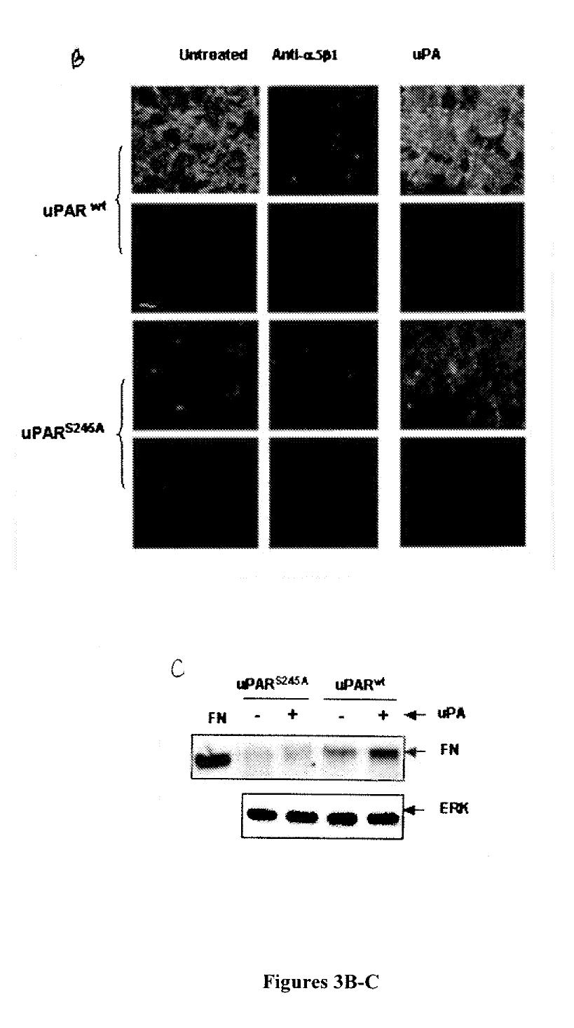

It has been shown that re-expression of uPAR in cells that also express α5β1-integrin leads to activation of the integrin (Aguirre-Ghiso et al., Mol. Biol. Cell 12:863-879 (2001), which is hereby incorporated by reference in its entirety). It was therefore tested whether the impaired binding observed for suPAR^{S245A} to purified α5β1-integrin in vitro translates into a loss of its ability to activate α5β1-integrin when expressed in cells. A glycolipid-anchored uPAR^{S245A} was generated by site-directed mutagenesis. HEK293 cells, which express α5β1-integrin (mean fluorescence 17.3) were transfected with a plasmid (pcDNA3.1) encoding either uPAR^{wt} or uPAR^{S245A}. Expression levels of uPAR^{S245A} and uPAR^{wt}, examined by FACS analysis (FIG. 3A, bottom panel) or Western blot were very similar. The activation state of the endogenous β1 integrin in these cells was assessed by FACS analysis using a conformation sensitive HUTS-4 antibody, which recognizes the active state of β1-integrin. In vector transfected HEK293 cells, half (50.9%) of the population had fluorescence intensity above the median (FIG. 3A, left panel). Treatment of these cells with Mn⁺⁺, an established activator of (31 integrins (Luque et al., J. Biol. Chem. 271:11067-11075 (1996), which is hereby incorporated by reference in its entirety), increased the population with fluorescence above the median to 86.2% (FIG. 3A, second panel from the left). In HEK293 cells expressing uPAR^{wt}, 74.4% of the population was above the median (FIG. 3A, third panel), a value similar to that of the Mn⁺⁺-treated vector transfected cells, while the uPAR^{S245A}-expressing cells had even fewer cells with active β1-integrins (44.6%) than the vector control (FIG. 3A, right panel). A similar difference in β1-activation produced by uPAR^{wt} and uPAR^{S245A} was found in uPAR^{wt} and uPAR^{S245A} transfected D-Hep3 cells.

To examine whether the activated β1-integrin subunit formed an active heterodimer specifically with the α5, and thus produced activated α5β1-integrin, fibronectin binding and fibronecting fibril formation on the uPAR-transfected cells were examined using immunofluorescence. Twenty-four hrs after transfection, the cells were plated on cover slips, incubated overnight with medium with 10% FBS and, then, for the next 24 hrs, with medium with fibronectin-depleted FBS, and supplemented with 30 μg/ml of human fibronectin with or without 10 μg/ml of α5β1-integrin blocking antibody. In addition, since it was previously shown that pro-uPA binding increased the signaling capacity of uPAR (Aguirre-Ghiso et al., J. Cell Biol. 147:89-104 (1999), which is hereby incorporated by reference in its entirety), cover slips with uPAR^{wt} or uPAR^{S245A} transfected cells were incubated for 24 hrs in plasminogen-depleted FBS with 15 nM pro-uPA. Cell-associated fibronectin smf fibronectin fibrils were detected by immunofluorescence and cell nuclei were identified by DAPI staining FIG. 3B shows that uPAR^{wt} transfected HEK293 cells had much more surface-bound fibronectin and fibrils than uPAR^{S245A}-transfected cells (FIG. 3B, top left panel) and anti-α5β1-blocking antibodies reduced the fluorescence to a barely detectable level (FIG. 3B, top middle panel), indicating a specific binding to and activation of the α5β1-integrin. Incubation of cells with pro-uPA, which were shown (Aguirre-Ghiso et al., J. Cell Biol. 147:89-104 (1999); Pluskota et al., Blood 101:1582-1590 (2003); Wei et al., Mol. Biol. Cell 12:2975-2986 (2001); Jo et al., J. Biol. Chem. 280:17449-17457 (2005), which are hereby incorporated by reference in their entirety) to increase uPAR interaction with the integrin, strongly increased the level of cell-bound fibronectin and fibrils in uPAR^{wt} cells (FIG. 3B, top right panel). In contrast, uPAR^{S245A}-transfected cells had on their surface barely detectable levels of fibronectin (FIG. 3B, lower left panel) and the integrin-blocking antibodies or pro-uPA had very small impact on the binding, suggesting that the observed binding may not be α5β1-specific. In ˜10% of uPAR^{wt}-transfected and pro-uPA-treated cells, fibronectin was organized into fibrils. The difference in fibronectin-binding was not due to a difference in fibronectin-production, because both uPAR^{wt}- and uPAR^{S245A}-transfected cells produced similar levels of fibronectin in Western blot analysis and the medium was supplemented with exogenous fibronectin. Using similar incubation conditions, the level of DOC-insoluble fibronectin-fibrils was compared in cells transfected either with uPAR^{wt} or uPAR^{S245A}. As shown in FIG. 3C, cells transfected with uPAR^{wt} had an easily detectable level of DOC-insoluble fibronectin, and treatment of cells with pro-uPA increased the level by ˜3 fold. In contrast, cells transfected with uPAR^{S245A} produced a very small amount of DOC-insoluble fibronectin that was unaffected by pro-uPA treatment of the cells. These results corroborate the immunofluorescence findings that, under similar conditions, show a reduced ability of uPAR^{S245A} to activate α5β1-integrin, bind fibronectin, and form fibrils (FIG. 3B).

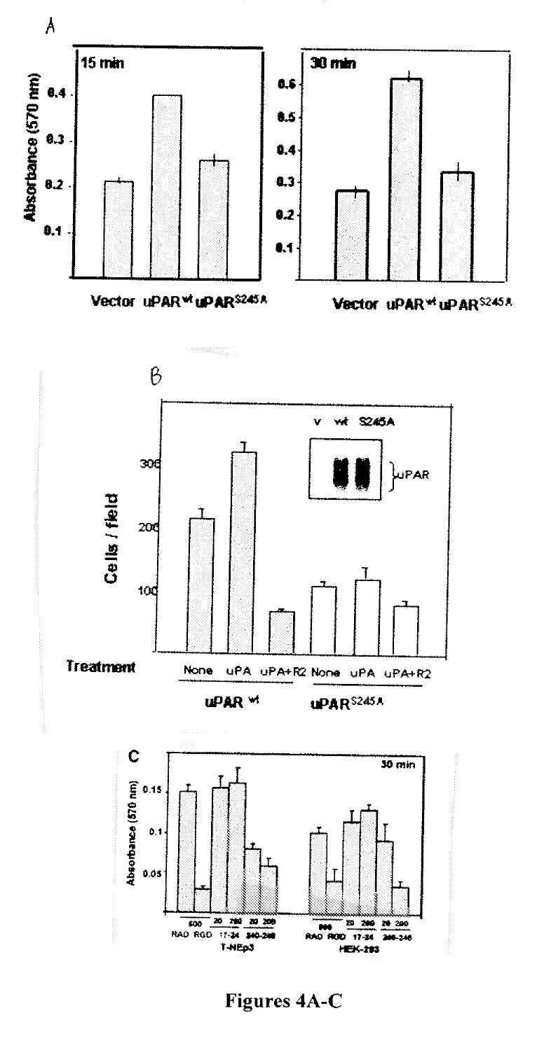

Another indication of α5β1-integrin activation is the enhanced ability of cells to adhere to fibronectin. To compare the effect of uPAR^{wt} and uPAR^{S245A} expression on cell adhesion to fibronectin, HEK293 cells were transiently transfected with pcDNA3.1 vector alone or with a plasmid coding for either uPAR^{wt} or uPAR^{S245A}, and the cells were tested for adhesion to fibronectin. As evident from FIG. 4A, compared to vector transfected cells, the adhesion of cells transfected with uPAR^{wt} was 1.9-fold greater at 15 min of incubation and 2.3 fold greater at 30 min. The increase in adhesion for cells transfected with uPAR^{S245A} over vector transfected cells was only 1.2-fold at both 15 and 30 min.

It has previously been shown that pro-uPA binding to uPAR strengthens the signaling cascade leading to ERK activation (Aguirre-Ghiso et al., J. Cell Biol. 147:89-104 (1999); Blasi et al., Nat. Rev. Mol. Cell. Biol. 3:932-943 (2002), which are hereby incorporated by reference in their entirety), suggesting that it may increase uPAR/integrin interaction. Since the binding affinity of uPA for suPAR^{wt} and suPAR^{S245A} is comparable (Table 1), the effect of pro-uPA on adhesion to fibronectin was directly compared. HEK293 cells transfected with uPAR^{wt} or uPAR^{S245A} were thus pre-incubated with 10 nM pro-uPA and inoculated on fibronectin-coated wells. While this treatment significantly (p=0.00) increased adhesion of cells expressing uPAR^{wt}, it had no significant effect on the adhesion of uPAR^{S245A} expressing cells (p=0.14) despite their similar uPAR expression levels (FIG. 4B, inset). Furthermore, cells transfected with uPAR^{wt} and treated with pro-uPA spread rapidly (within 15 min) when plated on fibronectin, producing multiple lamellipodia—a phenomenon that was not observed for uPAR^{S245A}-transfected cells. Importantly, treatment of the pro-uPA-pretreated uPAR^{wt} expressing cells with anti-uPAR antibodies reduced adhesion by ˜80%, whereas a reduction of only 35% was observed for identically treated, uPAR^{S245A}-expressing cells. This shows that complex-formation with uPA enhances the uPAR^{wt} dependent cell-adhesion. The functional impairment of integrin activation introduced by the uPAR^{S245A} mutation is not neutralized by the complex formation with uPA, arguing that the enhanced adhesion to fibronectin is governed by the uPAR/integrin interplay per se. This conclusion was further confirmed by an experiment in which the effect of disruption of uPAR/integrin interaction by the 240-248 peptide on the adhesion to fibronectin was tested. This was done both in T-HEp3 cells, which constitutively express high uPAR and adhere avidly to fibronectin (Aguirre-Ghiso et al., J. Cell Biol. 147:89-104 (1999), which is hereby incorporated by reference in its entirety), and in uPAR^{wt}-transfected HEK293 cells. As a control for blocking of adhesion, an RGD peptide was used, which is known for its ability to interfere with α5β1-integrin interaction with its fibronectin-ligand (Nishida et al., Invest. Opthalmol. Vis. Sci. 29:1820-1825 (1988), which is hereby incorporated by reference in its entirety). An inactive RAD peptide was used for comparison. As shown in FIG. 4C, 500 μM of the RGD peptide blocked adhesion of both T-HEp3 and HEK293-uPAR^{wt} cells to fibronectin. Incubation of both cell types with either 20 or 200 μM of peptide 240-248 reduced adhesion to fibronectin while peptide 17-24 had no effect.

The loss of integrin activation suggests that a single amino acid mutation in domain III of uPAR may abrogate the physical association between uPAR and integrin necessary for this functional interaction. To test this directly, the effect of the S245A mutation on uPAR/integrin co-immunoprecipitation was examined. D-HEp3 cells were used for this set of experiments, because it was shown previously that re-expression of the uPAR^{wt} returns these cells to a tumorigenic state (Aguirre-Ghiso et al., Mol. Biol. Cell 12:863-879 (2001), which is hereby incorporated by reference in its entirety). Cells were transiently transfected with the plasmid pcDNA3.1 (vector control), or plasmids encoding uPAR^{wt} or uPAR^{S245A}. Transfection efficiency was determined using pIRES2-EGF plasmid and was 30-40%. Cells were surface biotinylated, lysed, and immunoprecipitated with antibody to either α5β1-integrin or with anti-uPAR antibody to domain I (R3) (Ronne et al., FEBS Lett. 288:233-236 (1991), which is hereby incorporated by reference in its entirety), separated by PAGE and blotted with streptavidin. Association of uPAR with the integrin was quantified by scanning the resulting bands and expressing the intensity of uPAR band immunoprecipitated with the α5β1-integrin as percent of total uPAR precipitated by R3 anti-uPAR antibody. In the experiment shown in FIGS. 5A-B, 32% of the total uPAR^{wt}, and only 7% of uPAR^{S245A}, co-immunoprecipitated with the anti-α5β1-integrin. Only 3% of co-immunoprecipitation was observed in vector-control cells, supporting the notion that a certain threshold level of uPAR expression is required for uPAR/integrin interaction. This experiment was repeated 6 times, using additional anti-uPAR and anti α5β1-antibodies and it was found that although the percent of uPAR^{wt} that associated with α5β1-integrin varied (20% to 84%), the strong reduction in uPAR^{S245A} co-immunoprecipitation was always maintained. It was also shown that the preformed uPAR/integrin complex could be disrupted by incubation of the HEK293 expressing uPAR^{wt} cell lysates with 20 μM of 240-248 peptide, but not with the same concentration of the S245A mutant peptide (FIG. 5B).

### Example 17

### Cells Expressing uPAR^{S245A} Have Impaired ERK Activation and Lose their Ability to Grow In Vivo

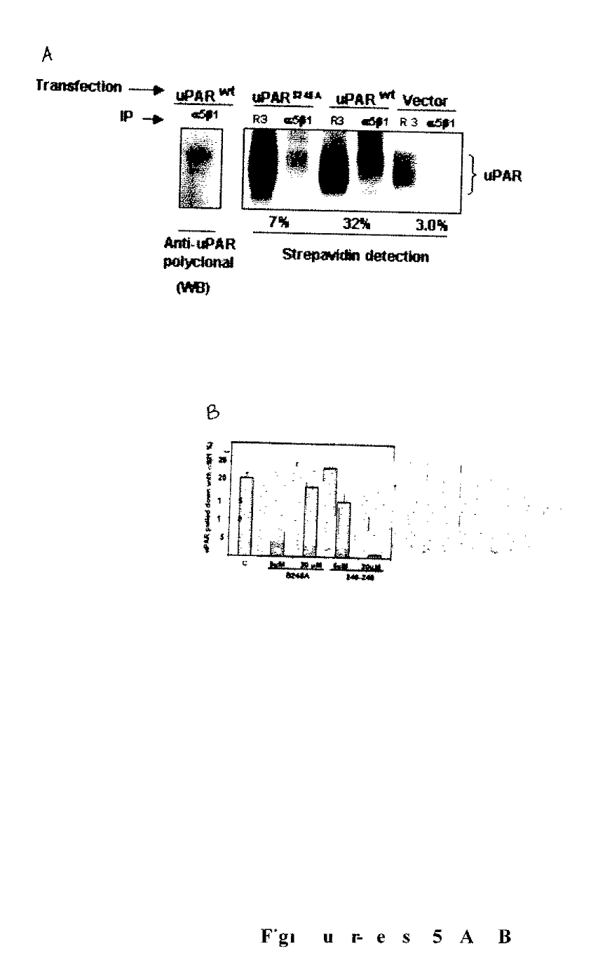

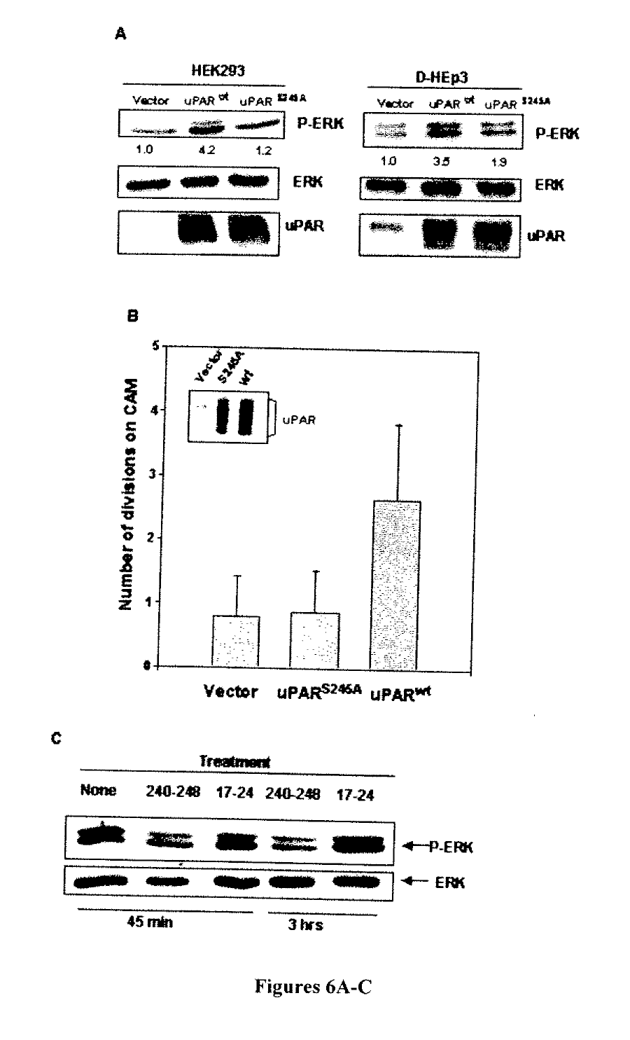

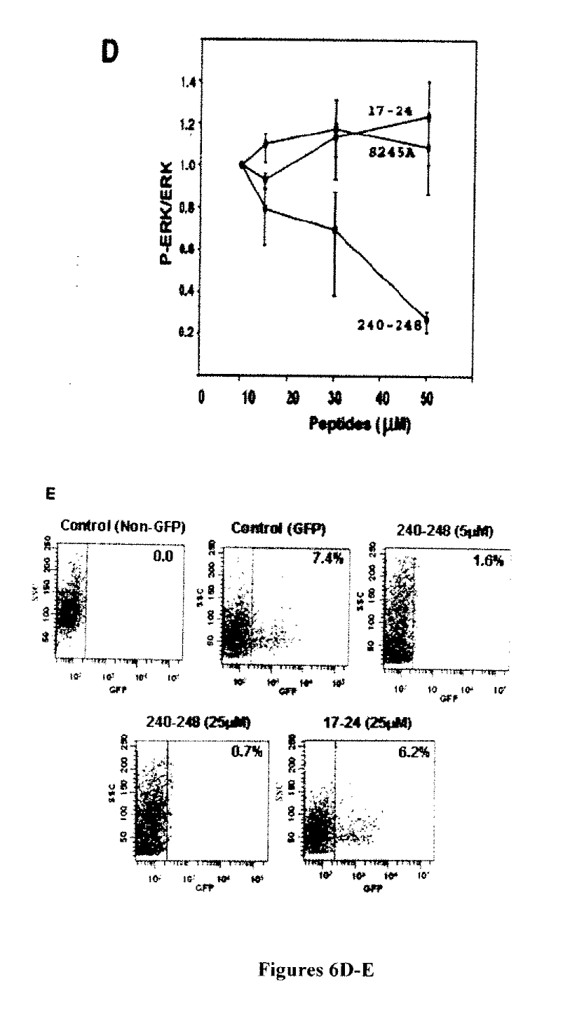

One of the functional hallmarks of the uPAR/integrin interaction is the initiation of a signaling pathway that leads to high levels of phosphorylated ERK allowing cancer cells to form tumors (Aguirre-Ghiso et al., J. Cell Biol. 147:89-104 (1999); Aguirre-Ghiso et al., Cancer Res. 63:1684-1695 (2003), which are hereby incorporated by reference in their entirety). It was thus tested whether the compromised activation of α5β1-integrin by uPAR^{S245A} is accompanied by a loss of ERK activation. D-HEp3 and HEK293 cells were transiently transfected with either pcDNA3.1 vector alone, or vectors encoding uPAR^{wt} or uPAR^{S245A}, and analyzed for uPAR expression as well as total and P-ERK content. The expression of uPAR^{wt} increased the ratio of P-ERK to ERK 4.2 fold in HEK293 cells relative to vector control and 3.5 fold in D-HEp3 cells. Despite a comparable expression level, uPAR^{S245A} transfection caused only a 1.2 and 1.9 fold increase in P-ERK to ERK ratio, respectively, in the two cell lines (FIG. 6A).

It has previously been shown that high ERK activity is required for in vivo growth of tumors (Ossowski et al., Curr. Opin. Cell Biol. 12:613-620 (2000), which is hereby incorporated by reference in its entirety). This implies that cells expressing uPAR^{S245A} with reduced ability to activate ERK should be restricted in their in vivo growth. To directly test this assumption, dormant D-HEp3 cells (Ossowski et al., Cell 35:611-619 (1983), which is hereby incorporated by reference in its entirety) were transiently transfected with vector alone, uPAR^{wt}, or uPAR^{S245A} (transfection efficiency was approximately 40% in all cases leading to very similar expression levels) (FIG. 6B, inset). Thirty-six hours post transfection, the cells were inoculated on CAMs and incubated for 4 days, the CAMs were excised, and the tumor cells counted. In accordance with previous experiments (Aguirre-Ghiso et al., Mol. Biol. Cell 12:863-879 (2001), which is hereby incorporated by reference in its entirety), D-HEp3 cells transfected with uPAR^{wt} completed more than 2 divisions in 4 days on CAM (median 2.2, mean 2.7 divisions) compared to less than 1 division for the mock-transfected cells (median 0.6, mean 0.8). More importantly, uPAR^{S245A}-transfected cells exhibited no growth stimulation above the vector-transfected cells (from median 0.6 to 0.8 and mean 0.8 to 0.9, p=0.75).

The above experiments suggest that impaired signaling to ERK is reflected in diminished ability to proliferate in vivo suggesting that the uPAR/integrin interaction may represent a potential target for therapy. Therefore, it was tested whether treatment of cells with the peptide 240-248 had any affect on the uPAR/integrin interaction and ERK activity. In one set of experiments T-HEp3 cells, serum-starved overnight, were treated with 25 μM of peptides 240-248 and 17-24 for 10, 45, and 180 min. Ten minute treatment produced only a minor effect. FIG. 6C shows that after 45 and 180 min of treatment only the 240-248 peptide produced a strong reduction in P-ERK level, while the 17-24 peptide had minimal or no effect on P-ERK. The effect of increasing concentrations of peptides 240-248, S245A, and 17-24 on P-ERK content after 1 hr of treatment was also tested (FIG. 6D), and it was found that peptide 240-248 reduced P-ERK level in a dose-dependent fashion between 5 and 40 μM, while the two other peptides had minor and inconsistent effects.

In an alternative approach to target ERK, T-HEp3 cells were used in which ERK activity was linked to GFP expression (Aguirre-Ghiso et al., Cancer Res. 63:1684-1695 (2003), which is hereby incorporated by reference in its entirety). This was achieved by stably expressing in HEp3 cells two constructs coding for an ELK-GAL4 fusion protein and for GAL4-UAS driven GFP. When ERK is active it phosphorylates Elk (in the Elk-GAL), increasing its association with the GAL4-UAS and transactivation of GFP expression. T-ELK cells were serum-starved overnight and incubated with peptide 240-248 at a concentration of 5 and 25 μM and a negative control peptide 17-24 at 25 μM for 38 hrs. As shown in FIG. 6E, FACS analysis showed that the population of GFP-positive cells (7.4% of total) was reduced to 1.6 and 0.7% by 5 and 25 μM of peptide 240-248, respectively. The negative-control peptide 17-24 reduced the population only slightly (to 6.2%).

It is reported here that functional interaction between uPAR and α5β1-integrin depends on a stretch of amino acid residues located within domain III of uPAR and the side-chain of S245 in particular is indispensable for these interactions. The identified sequence is located on the large outer surface in the 3-domain crystal structure of uPAR (Llinas et al., EMBO J. 24:1655-1663 (2005); Huai et al., Science 311:656-9 (2006), which are hereby incorporated by reference in their entirety) distinct from the central uPA-binding cavity. The engagement of uPAR in lateral interactions with integrins on the cell surface is well established (Ossowski et al., Curr. Opin. Cell Biol. 12:613-620 (2000); Chapman, Curr. Opin. Cell Biol. 9:714-724 (1997), which are hereby incorporated by reference in their entirety) and the binding site for uPAR has been identified on several integrins (Kugler et al., Curr. Pharm. Des. 9:1565-1574 (2003), which is hereby incorporated by reference in its entirety). However, the site on uPAR that binds α5β1-integrin has not been identified. It has previously been shown that uPAR/α5β1-integrin interaction initiates a signaling cascade that leads to ERK activation crucial for cancer cell growth in vivo (Aguirre-Ghiso et al., Mol. Biol. Cell 12:863-879 (2001), which is hereby incorporated by reference in its entirety). Importantly, this interaction is considered to be a potential target-site for anticancer therapy, because disruption of the uPAR/α5β1-integrin-interaction forces cancer cells into a state of dormancy.

The convergence of the experimental results described herein provides strong support to the conclusion that the 240-248 stretch of amino acids in uPAR is indeed representing at least a part of the functional binding site for the α5β1-integrin. In the dot-ELISA only 1 (peptide 240-248) out of 5 peptides tested showed binding activity. This peptide bound only weakly to another purified integrin of the β1-family, α3β1, suggesting specificity of the newly discovered site of interaction. However, the remarkable observation that a single amino acid substitution (S245A) in this synthetic peptide 240-248 completely eliminated its ability to bind the integrin lends credibility to its specific role in integrin interaction.

Full-length, recombinant uPAR proteins with single amino acid mutations were also tested within the stretch defined as the site of interaction by the synthetic peptide. It was found that several mutations within the 240-248 region, including mutation of S245A, disabled the integrin-binding ability of uPAR. A mutation in domain I of uPAR (E33A) had no effect. It was concluded that the loss of integrin binding is not caused by gross aberrant folding of the recombinant uPAR^{S245A}, since this mutant binds uPA with similar affinity as uPAR^{wt} (Table 1) and also binds several monoclonal anti-uPAR antibodies that recognize different conformation-dependent epitopes with comparable kinetic rate constants as determined for uPAR^{wt}. This and other published results showing that an anti-uPAR antibody that recognizes an epitope in domain III of uPAR (Ronne et al., FEBS Lett. 288:233-236 (1991); Hansen et al., Biochem. J. 380:845-57 (2004), which are hereby incorporated by reference in their entirety) disrupts the uPAR/integrin interaction and reduces the signal to ERK (Aguirre-Ghiso et al., Cancer Res. 63:1684-1695 (2003), which is hereby incorporated by reference in its entirety) fueled further inquiry into the functional relevance of the newly identified sequence.

To probe the biological relevance of the identified interaction site, cells with low or no endogenous uPAR were transfected with uPAR^{S245A} expressing plasmid. The loss of in vitro binding of the purified α5β1-integrin to uPAR^{S245A} would predict that the two receptors also lose their ability to interact in vivo, when present on the surface of cells. Indeed, it was found that anti-α5β1 antibody pulled down 32% of the total cell uPAR^{wt} while only a very small fraction (7% or less) of the total uPAR^{S245A} was pulled down by anti-α5β1 antibody (FIGS. 5A-B). Since it was shown that the expression levels of wild type and mutated uPAR are similar, and that the mutated receptor is properly localized to the cell surface (FIG. 3A), the loss of its association with the integrin can be directly ascribed to the S245A and, consequently, 5245 must participate in the in vivo interaction of the two proteins.

It was previously established that in cells expressing high levels of uPAR, such as cancer cells, the uPAR/α5β1-integrin interaction is responsible for integrin activation (Aguirre-Ghiso et al., Mol. Biol. Cell 12:863-879 (2001), which is hereby incorporated by reference in its entirety) as determined by uPAR-induced fibronectin-fibrillogenesis and increased cell adhesion to fibronectin (Aguirre-Ghiso et al., J. Cell Biol. 147:89-104 (1999), which is hereby incorporated by reference in its entirety). The downstream effect of this interaction is activation of ERK, which is further enhanced by pro-uPA binding to uPAR. To directly compare the ability of uPAR^{wt} and uPAR^{S245A} to activate integrin, a conformation-sensitive “reporter” antibody HUTS-4 was chosen, which recognizes an epitope in the hybrid domain (residue 355-425) of the β1-subunit (Luque et al., J. Biol. Chem. 271:11067-11075 (1996), which is hereby incorporated by reference in its entirety). This epitope is unmasked by an “outside in” activation of 131-integrins by Mn⁺⁺ or stimulatory antibodies (Luque et al., J. Biol. Chem. 271:11067-11075 (1996), which is hereby incorporated by reference in its entirety) that cause a downward movement of an α7 helix region of the βA domain and “swing-out” of the hybrid region of the β1-subunit (Mould et al., J. Biol. Chem. 278:17028-17035 (2003), which is hereby incorporated by reference in its entirety). It was postulated that a putative interaction between uPAR and the surface loop in the β-propeller of the α-subunit of integrin might expose the HUTS-4 epitope. Indeed, as shown (FIG. 3A), expression of uPAR^{wt}, but not uPAR^{S245A}, increased HUTS-4 binding with ˜86% of the efficacy of the MnCl₂ treatment, suggesting that uPAR may induce similar conformational change.

A more definitive indication of functional activation of α5β1-integrin was derived from testing of uPAR^{wt} induced fibronectin-binding to cell-surface and its enhancement by pro-uPA, which not only increased binding but induced fibrillogenesis in ˜10% of cells. Binding of fibronectin was almost completely prevented when cells were treated with blocking anti-α5β1-antibody, indicating that the observed uPAR-induced changes in fibronectin-binding are specific for α5β1-integrin. In contrast, cells that expressed uPAR^{S245A} bound barely detectable amounts of fibronectin, which remained unchanged upon treatment with α5β1-blocking antibodies or pro-uPA. The significance of the uPAR/α5β1 interaction in activating the α5β1-integrin was further substantiated by the experiments showing induced fibronectin-fibrillogenesis, a sign of active α5β1-integrins only in cells transfected with uPAR^{wt}, but not with uPAR^{S245A} (FIG. 3C). This difference in integrin activation was evidenced also in cell adhesion experiments, which showed that only uPAR^{wt} increased adhesion to fibronectin, that this adhesion was enhanced by pro-uPA treatment, and that monoclonal antibody to uPAR domain III blocked adhesion most likely by disrupting uPAR/integrin interaction and “de-activating” the integrin (FIGS. 4A-C). Moreover, peptide 240-248, but not peptide 17-24, was able to disrupt adhesion to fibronectin (FIGS. 4A-C). Only negligible effects were observed in cells expressing uPAR^{S245A}. The finding that adhesion to fibronectin is enhanced by uPAR and pro-uPA is in conflict with some published results (Wei et al., J. Cell Biol. 168:501-511 (2005); Simon et al., J. Biol. Chem. 275:10228-10234 (2000), which are hereby incorporated by reference in their entirety). Unless caveolin was expressed, uPAR expression blocked binding to fibronectin (Simon et al., J. Biol. Chem. 275:10228-10234 (2000), which is hereby incorporated by reference in its entirety) and later (Wei et al., J. Cell Biol. 168:501-511 (2005), which is hereby incorporated by reference in its entirety) that uPAR shifted the RGD-dependence of such interactions. uPA/uPAR was found to enhance binding to fibronectin (Aguirre-Ghiso et al., J. Cell Biol. 147:89-104 (1999); Liu et al., Cancer Cell 1:445-457 (2002), which are hereby incorporated by reference in their entirety). In addition, it was found that no loss of RGD-dependence of fibronectin binding in presence of uPAR. Although these differences remain unresolved, evidence for the interpretation that the interaction between the 240-248 sequence of uPAR and α5β1-integrin caused the latter to undergo activation.

The recently solved uPAR structure (Llinas et al., EMBO J. 24:1655-1663 (2005); Huai et al., Science 311:656-9 (2006), which are hereby incorporated by reference in their entirety) allowed the mapping of the newly identified integrin-binding sequence to a large surface on the “back” of the protein which is distinct from the uPA binding cavity (FIGS. 1G-H). It has been proposed (Llinas et al., EMBO J. 24:1655-1663 (2005), which is hereby incorporated by reference in its entirety) that binding of uPA or its growth-factor-like domain to the central cavity does not induce dramatic conformational changes in uPAR. This may thus allow the integrin binding to occur irrespective of the receptor occupancy with uPA. While this gross model of the uPAR-integrin complex seems plausible, it must be considered that uPAR is a relatively small modular protein folded into an almost globular structure, the integrin is large and, according to current model, becomes activated through a switchblade type unbending. It has been estimated (Tarui et al., J. Biol. Chem. 276:3983-3990 (2001), which is hereby incorporated by reference in its entirety) that the extended (activated) form of the integrin positions the RGD-binding site ˜200 Å from the plasma membrane. Since an uPAR binding site has been mapped to the β-propeller of several integrins (Simon et al., J. Biol. Chem. 275:10228-10234 (2000); Kugler et al., Curr. Pharm. Des. 9:1565-1574 (2003), which are hereby incorporated by reference in their entirety) a “switchblade” type activation by uPAR would not be feasible, unless the proteins are presented in “trans” on neighboring cells. A “trans uPAR/integrin” interaction has been described for other integrins (Tarui et al., J. Biol. Chem. 276:3983-3990 (2001), which is hereby incorporated by reference in its entirety) and it has been shown that suPAR can, by binding to the α5β1-integrin on the surface of cells, induce ERK activation (Aguirre-Ghiso et al., Mol. Biol. Cell 12:863-879 (2001), which is hereby incorporated by reference in its entirety), demonstrating the possibility of a functional interaction “in the trans configuration.” Alternatively, it is possible that, in addition to an extended active conformation, a “primed” partially bent state in which the integrins are capable of ligand binding, also exists (Mould et al., Curr. Opin. Cell Biol. 16:544-551 (2004), which is hereby incorporated by reference in its entirety). Recently, Adair et al. (J. Cell Biol. 168:1109-1118 (2005), which is hereby incorporated by reference in its entirety), using a transmission electron microscope has shown that a stable complex of Mn⁺⁺-bound extracellular-domain of αVβ3-integrin with fibronectin-type III domains 7-10 displayed compact triangular shape, indicative of bent conformation. Others have earlier suggested that straightening is not required to render integrin competent to bind physiological ligands (Butta et al., Blood 102:2491-2497 (2003); Calzada et al., J. Biol. Chem. 277:39899-39908 (2002), which are hereby incorporated by reference in their entirety).

Another group has identified an αVβ3-integrin binding sequence on domain II of uPAR (Degryse et al., J. Biol. Chem. 280:24792-24803 (2005), which is hereby incorporated by reference in its entirety), which was shown to harbor a short chemotactic sequence (GEEG). This peptide could induce migration in an αVβ3-integrin and uPAR-dependent manner at 1 pM. Another “chemokine” residing in the linker region between domain I and II of uPAR has previously been described (Resnati et al., EMBO J. 15:1572-1582 (1996), which is hereby incorporated by reference in its entirety). Unlike the sequence in domain III described herein, whereby a single mutation of S245A produces simultaneous drastic reduction of the interaction with the α5β1-integrin and its activation as well as a reduction in signal-transduction to ERK, the sequence in domain II of uPAR was shown to contain distinct interacting and chemotactic sequences (Resnati et al., EMBO J. 15:1572-1582 (1996), which is hereby incorporated by reference in its entirety). Binding of physiological integrin ligands is of relatively low affinity (K_{d} in μM) (Arroyo et al., J. Biol. Chem. 268:9863-9868 (1993), which is hereby incorporated by reference in its entirety), presumably to protect integrins from unintended activity. At present, it appears that the newly identified sequence in domain II of uPAR might belong to a new category of integrin activators different from the known physiological ligands of integrins.

The uPAR-sequence identified has a direct impact on the ability to design compounds that will disrupt the uPAR/integrin interaction and will also facilitate a more thorough mapping of the fine details of the uPAR-integrin interaction interface by a comprehensive alanine scanning mutagenesis. It is herein shown that uPAR and α5β1-integrin interact directly and that a mutation (S245A) that blocks integrin activation renders uPAR^{S245A} incapable of efficient ERK activation and, renders tumor cells that express it, incapable of in vivo growth. Moreover, proof of the principle indicating that effective targeting of this site of interaction reduces ERK activation and may force malignant cells into dormancy is herein provided.

### Example 18

### Compounds that Disrupt uPAR/Integrin Interaction for Induction and/or Maintenance of Tumor Dormancy and Prevention of Overt Metastases

The present invention relates to preventing cancer cells that have already spread from the primary tumor throughout the body from growing and forming detectable and life threatening metastases. Such cells remain undetectable for long periods of time, yet often they do reinitiate growth. During the period before growth reinitiation, the metastatic cells are dormant and the patient has no symptoms of disease.

A model of human oral cancer has been developed that can be experimentally forced from a highly malignant state, in which it produces invasive, metastatic cancer, into a state of dormancy. It has been found that the malignant cells have on their surface a protein, uPAR, which binds uPA and interacts with two additional surface proteins, integrin (α5β1) and the epidermal growth factor receptor (EGFR). When all of the components are present, their interaction sends a potent intracellular signal, which causes a series of enzymatic reactions. This leads to a very strong and persistent activation of growth promoting MAPK-ERK pathway while at the same time inhibiting the growth-arresting MAPK-p38. These reactions subsequently facilitate the assembly of insoluble fibronectin fibrils on the cell surface.

Previously, it was found that dormancy can be induced by treating cancer cells with an anti-uPAR antibody (R2), directed to an epitope located in domain III of uPAR, and disrupting the interaction between uPAR and α5β1 integrin. For several reasons, this is considered to be important for development of therapy that will prevent the conversion of occult micrometastases to overt metastases. First, the target, i.e., the interaction between uPAR-integrin, is tumor specific, because it occurs only when the urokinase receptor is present at a very high level, a characteristic of many malignant tumors. In fact, urokinase and urokinase level have been used to predict the clinical outcome in several cancer types, where it was found that the higher the concentration of these proteins on the surface of tumor cells, the worse the prognosis. In addition, the interaction (target) takes place on the cell surface making access of a therapeutic entity easier. Lastly, rather than targeting and killing dividing cells, the therapy disclosed herein will likely prevent emergence of overt metastases by maintaining dormancy. In order to be effective, such therapy would have to be used chronically; thus, a potential drug must have minimal toxicity.

Screening of the Library by Docking to uPAR and/or to the Integrin Binding Sequence

The in silico screening of a library of about 13,000 small molecules was conducted for possible binders to uPAR and the specific site on uPAR that binds integrin α5β1. The calculations used the software Autodock (v 3.05). The input describing the protein was prepared with the program Autodock Tools (ADT); it involved adding charges and non-bonded parameters to the protein structure file and orienting the protein to minimize the enclosing rectangle (using Dr. Mihaly Mezei's program Simulaid). The screening was driven by a script that Dr. Mezei wrote that runs the docking of several ligands on different CPUs of the cluster in parallel, allowing the full screening to be completed in a couple of weeks. The docked poses were sorted and filtered by Dr. Mezei's computer program Dockres and the top-scoring molecules were tested using several cell-based assays. Furthermore, promising ligands were entered into the ZINC database of (over 4.6×10⁶) small molecules, and commercially available analogs were found for further testing.

Screening of Compounds by Luciferase Assay

To test the effect of uPAR-integrin disruption by uPAR-docked compounds on ERK activation, T-HEp3 cells were transiently co-transfected with pFA-Elk1-fusion plasmid and pD700-luciferase plasmid in a 96-well plate. These plasmids report the state of ERK activation through luciferase activity level. After 24 h of transfection, cells were serum starved for 5 h and incubated with PD98059 (Calbiochem, San Diego, Calif.) (25 and 50 μM) and 5 μM of each compound prepared in DMEM. Following 16 h of incubation with compounds, the cells were lysed directly in Steady glo-luciferase lysis buffer (1:1) from Promega and read on Tecan Safire2™ using Magellan 6.0 software. Histographs representing the percent inhibition of Luc-Elk activity as readout for ERK reduction were generated.

Test of Toxicity of Compounds

T-HEp3 cells (2×10³/well) were inoculated in a 96-well plate and incubated for 24 h. The compounds being tested, diluted in DMEM, were added to a final concentration of 1, 2.5, 5, 7.5, 10, and 12.5 μM. After 24 h of incubation, the live cells were counted. The results are represented as growth inhibition (%), compared to untreated control.

Fibronectin (Fibronectin)-Fibrils Disruption

To test the effects of fibronectin-fibril disruption by the compounds, T-HEp3 cells were incubated in 5% fibronectin-depleted fetal bovine serum (FBS)/DMEM with 0, 2.5, 5, 7, and 10 μM of the compounds or anti-uPAR antibody (R2, 20 μg/ml) or anti-α5β1 antibody (BIIG2, 20 μg/ml) or MEK inhibitor (PD98059, 25 μM) as a negative control at room temperature for 15 min prior to plating them in chambered slides. Cells were allowed to attach for 1 h at 37° C., the medium was supplemented with 10 μg/ml of human fibronectin and the cells were incubated for 16 h. The cells were fixed with 3% paraformaldehyde for 20 min and the bound fibronectin was detected using rabbit anti-human fibronectin antibody and with FITC-conjugated secondary antibody.

Adhesion Assay

T-HEp3 cells were detached with 4 mM EDTA and incubated in suspension in DMEM with anti-uPAR antibody R2, 15 μg/ml or the compounds being tested at final concentrations of 15, 30, and 60 μM. The cells were inoculated (7×10⁴ cells per well) in triplicates in a 96-well plate pre-coated overnight with 0.4 μg/ml of fibronectin and blocked with 0.1% BSA for 1 h at 37° C. Following 20 min incubation at 37° C., the unattached cells were removed by aspiration and the cell monolayers were washed twice with PBS with CaCl₂ and MgCl₂, fixed with 1% glutaraldehyde, stained with 1% crystal violet for 10 min, washed, and dried. The wells were photographed and cells present in four fields/well (a minimum of 1200 cells) were counted.

Disruption of uPAR-α5β1 Integrin Complex

T-HEp3 cell lysates were pre-cleared with protein G-agarose beads preincubated with isotype-matched IgG for 45 min at 4° C. on a rolling platform in the presence of protease inhibitors (1 mM PMSF, 1 μg/ml aprotinin, 1 μg/ml leupeptin, 1 μg/ml pepstatin). The pre-cleared cell lysates (0.8 mg) were incubated for 1 hr at 4° C. with protein G-agarose beads to which 5 μg of anti-α5β1 (HAS), anti-uPAR(R3) antibodies, or isotype-matched IgG was bound (pull-down) and 2.5, 5, and 10 μM of the compounds being tested were added to the beads and incubated for 20 min at 4° C. The protein G-beads were washed twice with PBS and protease inhibitors (1 mM PMSF, 1 μg/ml aprotinin, 1 μg/ml leupeptin, 1 μg/ml pepstatin), and once with 0.1% Nonidet P-40 (NP-40) resuspended in 2×-Laemmli sample buffer, heated, separated on SDS-PAGE, transferred onto a PVDF membrane, probed either with rabbit anti-uPAR polyclonal antibody or streptavidin-HRP, washed and developed using ECL, and scanned with NIH image.

The precise site on uPAR that is involved in the interaction with the integrin has been identified and found to be responsible for signaling to ERK. Solving of the crystal structure of uPAR and the above identification of the 240-248 sequence in domain III as the binding site for the integrin permitted docking of a library of compounds using computational analysis.

Of the 68 compounds that docked (30 on uPAR and 38 on the 240-248 sequence), 10 compounds (5 on uPAR and 5 on the 240-248 sequence) showed inhibition of ERK activation when first tested by the luciferase assay. Among the 10 compounds, two compounds having the strongest ERK inhibitory activity (i.e., MS0012479 and MS0019128) (FIGS. 7A-B), were selected. The two compounds at 5 μM were as effective as PD98059, a commercial MEK inhibitor, at 25 μM (FIG. 8).

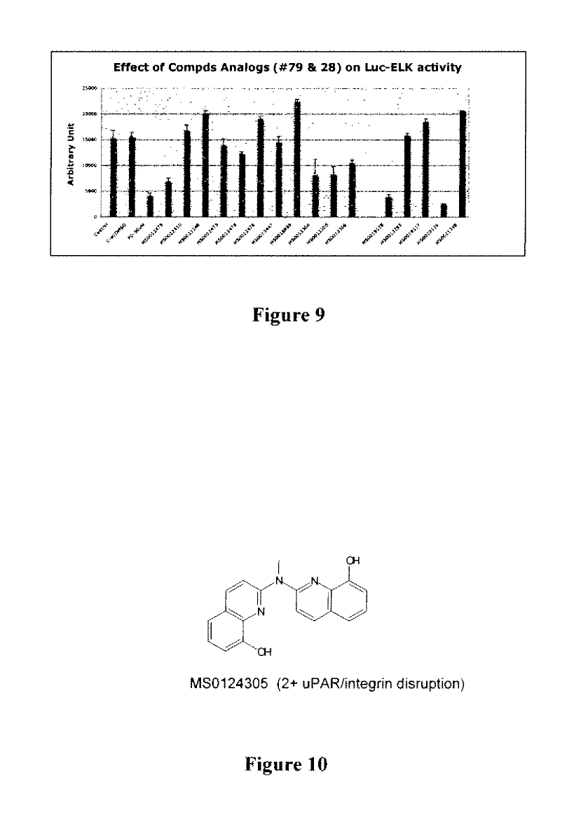

A structure activity relationship analysis of compounds MS0012479 and MS0019128 was performed. This was done with commercially available compounds and not through directed modification of chemical groups. Of the 10 analogs of compound MS0012479, four analog compounds (i.e., MS0012304, MS0012305, MS0012306 (strong) and MS0012476 (weak)), had ERK inhibitory activity. Of the 4 analogs of compound MS0019128, only one analog compound (i.e., MS0019126) had very strong ERK inhibitory activity (FIG. 9). The ERK inhibitory activity was tested using cells transiently transfected with the ELK-GAL-luciferase promoter/reporter construct.

Compounds MS0012479 and MS0019128 and their analogs were tested for fibronectin-fibril disruption. To quantify the effect of compounds on uPAR-integrin disruption, the cells were treated with the compounds (5 and 10 μM) and 16 h later revealed fibronectin fibrils using immunofluorescence. A total of 700 to 1150 cells were counted in 5 individual fields. Cells displaying fibronectin fibrils were then counted and expressed as a percentage of total cells. The mean obtained from the compound-treated cells was expressed as a percentage of control. The control had 90.2% of fibril positive cells (Table 2).

TABLE 2

| | Qualitative score | Quantification |
|---|---|---|
| Compound | (Degree of reduction) | (% of control) |
| MS0012479 | 3+ | 25.8 |
| MS0012305 | 2+ | 33.0 |
| MS0019126 | + | 66.0 |
| MS0012476 | +/− | Not evaluated |
| Ab blocking | 3+ | 24.1 |
| α5β1 integrin | | |

As shown in the above table, it was found that compounds MS0012479 and MS0012305 (FIG. 10) were effective in reducing the level of fibronectin-fibrils, indicating reduction in α5β1-integrin activation. Therefore, while seven compounds, i.e., MS0012479, MS0019128, MS0019126, MS0012304, MS0012305, MS0012306 and MS0012476, showed ERK inhibitory activity, only two compounds, i.e., MS0012479 and MS0012305, showed strong uPAR-integrin disruption.

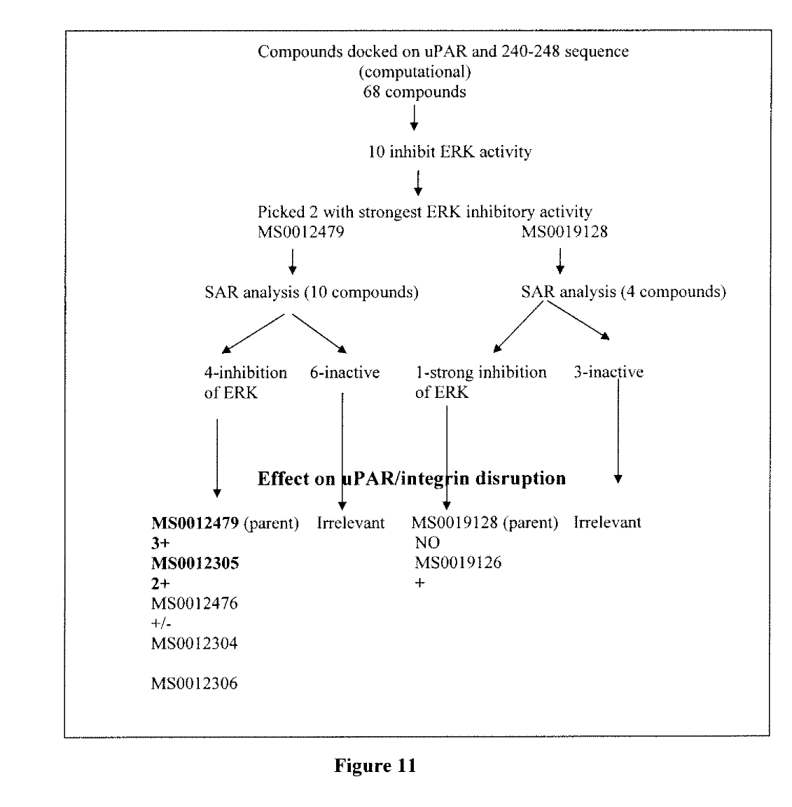

The flow chart in FIG. 11 summarizes how the compounds were selected.

### Example 19

### Effect of Compound MS0012479 on Tumor Growth In Vivo on Chorioallantoic Membrane (CAM)

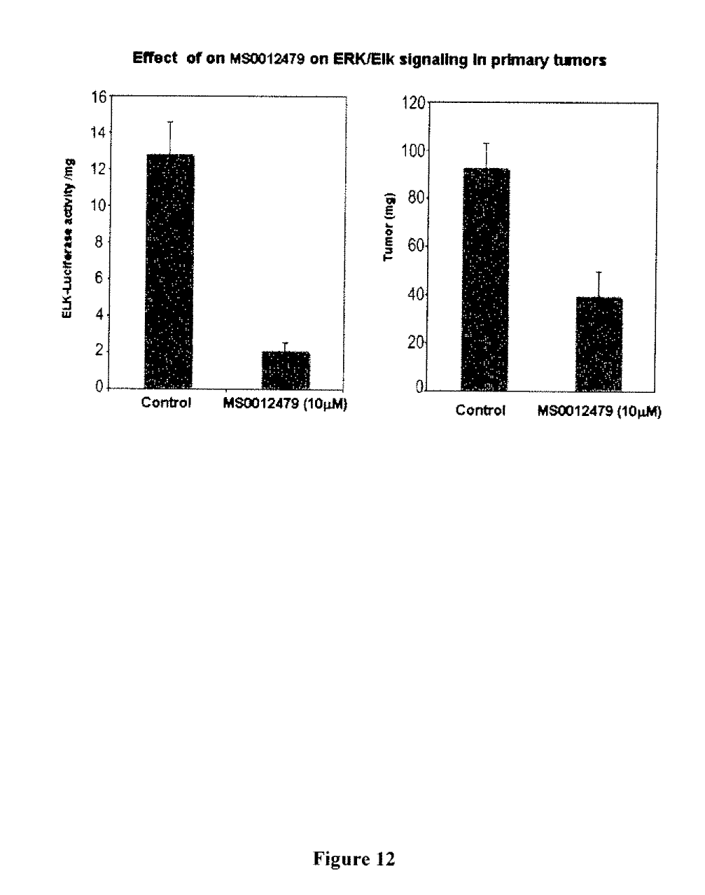

T-HEp3 cells stably transfected with pFA-Elk1-fusion plasmid and reporter pD700-luciferase plasmid (Stratagene, La Jolla, Calif.) were detached with EDTA, counted, resuspended at ˜8×10⁶ cells/ml in PBS with calcium and magnesium and either 0.5% DMSO (control) or 10 μM compound MS0012479. Cells (2.5×10⁵ per CAM) were inoculated in 30 μl, into a 8 mm Teflon ring placed on the CAM. Seven eggs were inoculated per group. The inoculated cells were treated either with 30 μl of PBS/DMSO or PBS/10 μM compound on 5 consecutive days at which time the tumors were excised, weighed, minced, lysed, and the luciferase activity was measured using Steady Glo (Promega, San Luis Obispo, Calif.). Results shown (FIG. 12) are tumor weight (right) and luciferase activity per mg of tumor (left). These results demonstrate that MS0012479 is effective in reducing tumor growth and ERK/ELK signaling.

Although the invention has been described in detail for the purpose of illustration, it is understood that such detail is solely for that purpose, and variations can be made therein by those skilled in the art without departing from the spirit and scope of the invention which is defined by the following claims.

What is claimed:

1. A method of inducing cancer cells into dormancy and treating cancer in a subject comprising:
administering to a subject an effective amount of a compound that disrupts interaction between urokinase plasminogen activator receptor and integrin, said compound having the following formula: 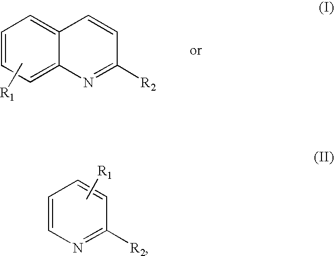
wherein
R₂ is 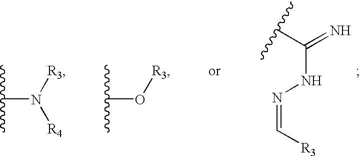
R₃ is 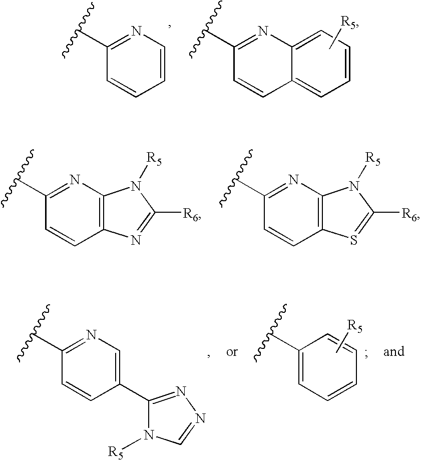
R₁, R₄, R₅, and R₆ are independently selected from the group consisting of hydrogen, —(CH₂)ₙOH, —(CH₂)ₙNH₂, —(CH₂)ₙNHCH₃, —(CH₂)ₙNHCH₂CH₃, —(CH₂)ₙ—COOH, halo, lower alkyl, haloalkyl, —(CH₂)ₙSH, lower thioalkyl, —CN, lower alkoxy, and 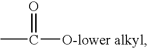
where n is an integer from 0 to 4, and pharmaceutically acceptable salts thereof, thereby inducing cancer cells into dormancy and treating cancer in the subject.

2. The method according to claim 1, wherein the compound has the following formula:

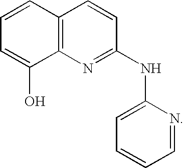

3. The method according to claim 1, wherein the compound has the following formula:

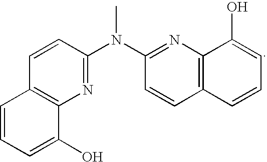

4. The method according to claim 1, wherein the compound has the following formula:

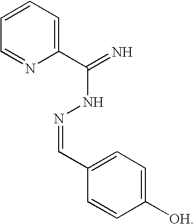

5. The method according to claim 1, wherein the compound has the following formula:

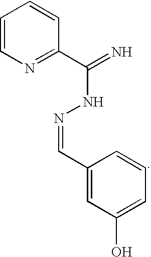

6. The method according to claim 1, wherein the compound is administered as part of a composition further comprising a pharmaceutically acceptable carrier.

7. The method according to claim 1, wherein said administering is carried out orally, topically, transdermally, parenterally, subcutaneously, intravenously, intramuscularly, intraperitoneally, by intranasal instillation, by intracavitary or intravesical instillation, intraocularly, intraarterially, intralesionally, by inhalation, or by application to mucous membranes.

8. The method according to claim 1, wherein the cancer is selected from the group consisting of head and neck squamous cell carcinoma, breast cancer, ovarian cancer, prostate cancer, colon cancer, squamous carcinoma of the skin, glioblastoma, endometrial carcinoma, gastric cancer, pancreatic cancer, renal cell carcinoma, squamous cell lung cancer, and bladder cancer.

9. The method according to claim 1, wherein the subject is a human.

10. A method of disrupting interaction between integrin and urokinase plasminogen activator receptor on cancer cells comprising:
contacting cancer cells with an effective amount of a compound having the following formula: 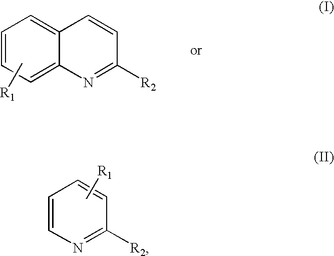
wherein
R₂ is 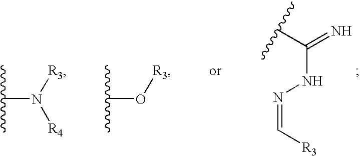
R₃ is 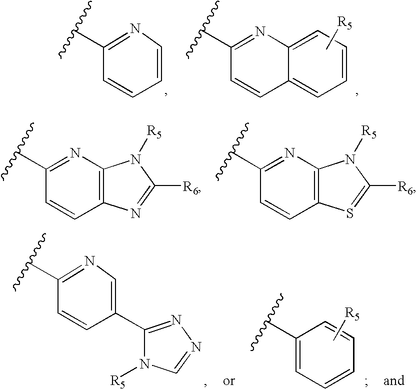
R₁, R₄, R₅, and R₆ are independently selected from the group consisting of hydrogen, —(CH₂)ₙOH, —(CH₂)ₙNH₂, —(CH₂)ₙNHCH₃, —(CH₂)ₙNHCH₂CH₃, —(CH₂)ₙ—COOH, halo, lower alkyl, haloalkyl, —(CH₂)ₙSH, lower thioalkyl, —CN, lower alkoxy, and 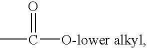
where n is an integer from 0 to 4, and pharmaceutically acceptable salts thereof.

11. The method according to claim 10, wherein the compound has the following formula:

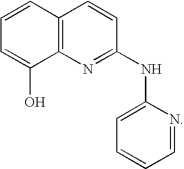

12. The method according to claim 10, wherein the compound has the following formula:

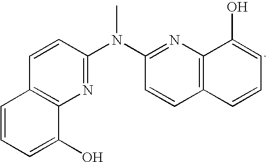

13. The method according to claim 10, wherein the compound has the following formula:

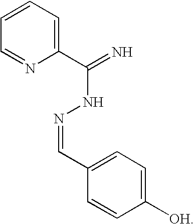

14. The method according to claim 10, wherein the compound has the following formula:

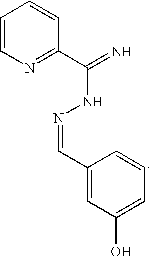

15. The method according to claim 10, wherein said contacting cancer cells is carried out in vitro.

16. The method according to claim 10, wherein said contacting cancer cells is carried out in vivo.

17. A method of screening for compounds effective in inducing tumor dormancy and treating cancer in a subject, said method comprising:
providing one or more candidate compounds;
contacting the one or more candidate compounds with urokinase plasminogen activator receptor under conditions effective to disrupt interaction between integrin and the urokinase plasminogen activator receptor; and
identifying candidate compounds which disrupt interaction between integrin and urokinase plasminogen activator receptor as compounds potentially effective in inducing tumor dormancy and treating cancer in a subject.

18. The method according to claim 17, wherein said contacting comprises contacting the one or more candidate compounds with a fragment of urokinase plasminogen activator receptor having the sequence of SEQ ID NO:9.

19. The method according to claim 17, wherein said contacting is carried out by computer simulation.

20. The method according to claim 17, wherein said contacting is carried out by physical contact.


SEQUENCE LISTING (9 sequences)

SEQ ID NO: 1 - DNA, 30 bp, Artificial
  primer sequence
gctgtgcaaccgcctcaatgtgccaacatg

SEQ ID NO: 2 - DNA, 30 bp, Artificial
  primer sequence
catgttggcacattgcggcggttgaacagc

SEQ ID NO: 3 - DNA, 16 bp, Artificial
  primer sequence
gagactttcctcattg

SEQ ID NO: 4 - DNA, 15 bp, Artificial
  primer sequence
aatgaggaaagtctc

SEQ ID NO: 5 - protein, 8 aa, Artificial
  synthetic peptide
CALGQDLC

SEQ ID NO: 6 - protein, 9 aa, Artificial
  synthetic peptide
LTEVVCGLD

SEQ ID NO: 7 - protein, 12 aa, Artificial
  synthetic peptide
AVTYSRSRYLEC

SEQ ID NO: 8 - protein, 11 aa, Artificial
  synthetic peptide
GRHQSLQCRSP

SEQ ID NO: 9 - protein, 9 aa, Artificial
  synthetic peptide
GCATASMCQ